(19) United States
(12) Patent Application Publication
(10) Pub. No.: US 2025/0000906 A1
(43) Pub. Date: Jan. 2, 2025

(54) TREATMENT OF MYXOID/ROUND CELL LIPOSARCOMA PATIENTS

(71) Applicant: SOTIO BIOTECH INC., Boston, MA (US)

(72) Inventor: Glen J. WEISS, Cambridge, MA (US)

(73) Assignee: SOTIO BIOTECH INC., Boston, MA (US)

(21) Appl. No.: 18/709,546

(22) PCT Filed: Nov. 15, 2022
(86) PCT No.: PCT/US2022/079885

§ 371 Date: May 13, 2024

Related U.S. Application Data
(60) Provisional application No. 63/279,797, filed on Nov. 16, 2021.

(51) Int. Cl. A61K 35/17; A61K 31/675; A61K 31/7076; A61K 39/00; C07K 16/30; G01N 33/574

(52) U.S. Cl. CPC A61K 35/17; A61K 31/675; A61K 31/7076; A61K 39/4611; A61K 39/4631; A61K 39/464474; C07K 16/303; G01N 33/57492; A61K 2239/13; A61K 2239/21; G01N 2474/20

(57) ABSTRACT

The present invention provides a method of treating a patient diagnosed with Myxoid/Round cell liposarcoma with an anti-GPC3 therapeutic agent. The present invention also relates to quantification of GPC3 expression in tissue samples of patients diagnosed with Myxoid/Round cell liposarcoma by an immunostaining assay and identification of GPC3 expression levels that correlate with selection of patients for administering the anti-GPC3 therapeutic agent.

### CROSS REFERENCE TO RELATED APPLICATIONS

This application claims the benefit of the filing dates of U.S. Provisional Application No. 63/279,797, filed Nov. 16, 2021, the entire contents of which are incorporated by reference herein.

### BACKGROUND OF DISCLOSURE

Sarcomas, including soft tissue sarcoma (STS), are rare group of malignancies of mesenchymal origin accounting for about 20% of all pediatric and about 1% of adult solid tumors (Abaricia & Hirbe, 2018; Hui, 2016). Liposarcoma (LPS), the most common type of STS, arises from adipose tissue and is a malignant neoplasm affecting fat differentiation. Myxoid/round cell liposarcoma (MRCLS) accounts for approximately 30% of LPS and tends to occur in slightly younger age group with peak incidence in the fourth decade of life.

Standard treatment for MRCLS consists of surgical resection for localized, primary disease. MRCLS is known for its sensitivity to radiation therapy and cytotoxic chemotherapy in comparison to the other LPS subtypes. However, despite the appropriate local treatment, about 40% of patients do relapse. Still, treatment options for patients with MRCLS continue to be poor and new therapy options are required for this high-medical need indication.

### SUMMARY OF DISCLOSURE

The present disclosure is based, at least in part, on the unexpected discovery that Glypican 3 (GPC3) can serve as a diagnostic biomarker and treatment target for MRCLS. Upon surveying the expression profile of GPC3 in solid tumor biopsies from cancer patients to better understand GPC3 positive tumor prevalence and for indication prioritization for an anti-GPC3 therapy, a sub-group of LPS, namely MRCLS, is identified as a relatively high GPC3 expressing patient population, utilizing new scoring rules to quantify the level of GPC3 expression accurately and reliably across multiple tissue samples and pathologists doing the scoring. This finding is unexpected given the complexity of staining patterns observed in various tumor tissue sections stained with an anti-GPC3 antibody (GC33) and suggests MRCLS to be prioritized and/or selected for treating with an anti-GPC3 therapeutic agent.

Accordingly, the present disclosure features, in some aspects, a method of treating a patient diagnosed with myxoid/round cell liposarcoma, the method comprising administering an anti-Glypican-3 (GPC3) therapeutic agent to the patient. In some embodiments, the patient is selected for treatment by diagnosing myxoid/round cell liposarcoma. In some examples, the myxoid/round cell liposarcoma expresses GPC3.

In some embodiments, the patient diagnosed with the myxoid/round cell liposarcoma is selected by immunostaining, for example, by immunohistochemistry (IHC) staining. In some examples, the patient diagnosed with the myxoid/round cell liposarcoma is selected if the patient has a cytoplasmic/membranous H-score of greater than 30.

In some embodiments, the patient may be diagnosed by a process comprising:
(a) obtaining a tissue section from a tumor biopsy sample, the section having a thickness between 3 μm and 15 μm,
(b) immunostaining preferably by IHC with an antibody that specifically binds to GPC3, more specifically using the antibody GC33,
(c) determining the cytoplasmic/membranous H-score, and
(d) selecting patients with an H-score of greater than 30 for the treatment.

In some embodiments, the selection may comprise immunostaining, for example, immunohistochemical staining of GPC3 in a tumor sample from the patient. The GPC3 expression level may be determined and compared to a predetermined threshold level of GPC3 expression. The patient is selected for treatment in case the patient has a GPC3 expression level equal or higher to the predetermined threshold level.

In some embodiments, the therapeutic agent comprises an anti-GPC3 binding domain, for example, an anti-GPC3 antibody, e.g., a full-length antibody or functional fragment thereof retaining binding to GPC3. In some instances, the therapeutic agent may comprise an anti-GPC3 antibody, an anti-GPC3 antibody-drug conjugate, an anti-GPC3 antibody-radionuclide conjugate, or a fusion protein of an anti-GPC3 antibody or antibody derivative binding to GPC3 with an anti-CD3 binding domain or an immunostimulatory polypeptide. In some examples, the therapeutic agent comprises genetically engineered hematopoietic cells expressing an anti-GPC3 chimeric receptor polypeptide (CAR), which may comprise:
(a) an extracellular binding domain binding to GPC3;
(b) a transmembrane domain; and
(c) a cytoplasmic signaling domain.

In some examples, the hematopoietic cell may further exogenously expresses a gene that improves viability and/or functionality of the hematopoietic cell in the solid tumor microenvironment. In some instances, the hematopoietic cells may have an improved glucose uptake activity as relative to a wild-type hematopoietic cell of the same type, whereas the hematopoietic cell exogenously expresses a glucose importation polypeptide. In some examples, the glucose importation polypeptide is a glucose transporter (GLUT) or a sodium-glucose cotransporter (SGLT). Examples include, but are not limited to, GLUT1, GLUT3, GLUT1 S226D, SGLT1, SGLT2, GLUT8, GLUT8 L12A L13A, GLUT11, GLUT7, and GLUT4.

In some instances, the hematopoietic cells may have a modulated Krebs cycle as relative to a wild-type hematopoietic cell of the same type, whereas the hematopoietic cell exogenously expresses a Krebs cycle modulating polypeptide. In some examples, the Krebs cycle modulating factor is an enzyme that catalyzes a reaction in the Krebs cycle. Examples include, but are not limited to, isocitrate dehydrogenase (IDH), malate dehydrogenase (MDH), or phosphoglycerate dehydrogenase (PHGDH). In some examples, the Krebs cycle modulating factor is an enzyme that uses a Krebs cycle metabolite as a substrate, for example, glutamic-oxaloacetic transaminase (GOT) or phosphoenolpyruvate carboxykinase 1 (PCK1). Alternatively, the Krebs cycle modulating factor may be an enzyme that converts a precursor to a Krebs cycle metabolite. Examples include, but are not limited to, phosphoserine aminotransferase (PSAT1), glutamate dehydrogenase (GDH1), glutamate-pyruvate transaminase 1 (GPT1), or glutaminase (GLS).

In some instances, the hematopoietic cells may have enhanced intracellular lactate concentrations relative to a wild-type hematopoietic cell of the same type, whereas the hematopoietic cell exogenously expresses a lactate-modulating polypeptide. In some examples, the lactate modulation polypeptide is monocarboxylate transporter (MCT), for example, MCT1, MCT2, or MCT4. In other examples, the lactate modulating polypeptide is an enzyme involved in lactate synthesis, e.g., lactate dehydrogenase A (LDHA). Alternatively, the lactate modulating polypeptide is a polypeptide that inhibits a pathway that competes for lactate-synthesis substrates, for example, pyruvate dehydrogenase kinase 1 (PDK1).

In any of the methods disclosed herein, the extracellular antigen binding domain is a single chain antibody fragment (scFv) that binds to a GPC3. In some examples, the scFv is derived from the GC33 antibody. In one example, the scFv may comprise (e.g., consists of) the sequence of SEQ ID NO: 2. In some examples, the anti-GPC3 CAR polypeptide may comprise a CD28 co-stimulatory domain, in combination with a CD28 transmembrane domain, a CD28 hinge domain, or a combination thereof (e.g., SEQ ID NO: 4). Alternatively, the anti-GPC3 CAR polypeptide may comprise a 4-1BB co-stimulatory domain (e.g., SEQ ID NO: 5), in combination with a CD8 transmembrane domain, a CD8 hinge domain, or a combination thereof (e.g., SEQ ID NO: 3). Alternatively or in addition, the anti-GPC3 CAR polypeptide may comprise a cytoplasmic signaling domain of (c), which may be a cytoplasmic domain of CD3ζ, preferably SEQ ID NO: 7, or FcεR1γ. In specific examples, the anti-GPC3 CAR may comprise the amino acid sequence of SEQ ID NO: 8 or SEQ ID NO: 9.

Any of the hematopoietic cells disclosed herein may be natural killer (NK) cells, macrophages, neutrophils, eosinophils, or T cells. In some examples, the hematopoietic cells are are T cells. In some instances, the expression of an endogenous T cell receptor, an endogenous major histocompatibility complex, an endogenous beta-2-microglobulin, or a combination thereof has been inhibited or eliminated in such T cells. In some examples, the hematopoietic cells may be derived from peripheral blood mononuclear cells (PBMC), hematopoietic stem cells (HSCs), or inducible pluripotent stem cells (iPSCs). In some examples, the hematopoietic cells are autologous to the patient. In other examples, the hematopoietic cells are allogeneic to the patient.

In some embodiments, the hematopoietic cells may comprise a nucleic acid or a set of nucleic acids, for example, a DNA molecule or a set of DNA molecules, which collectively comprises: (a) a first nucleotide sequence encoding the glucose importation polypeptide, the Krebs cycle modulating polypeptide and/or the lactate-modulating polypeptide; and (b) a second nucleotide sequence encoding the chimeric antigen receptor polypeptide. In some examples, the hematopoietic cells may comprise the nucleic acid, which comprises both the first nucleotide sequence and the second nucleotide sequence. In some instances, the nucleic acid may comprise a third nucleotide sequence located between the first nucleotide sequence and the second nucleotide sequence. The third nucleotide sequence may encode a ribosomal skipping site, an internal ribosome entry site (IRES), or a second promoter. In some examples, the third nucleotide sequence encodes a ribosomal skipping site, for example, a P2A peptide. In some examples, the nucleic acid or the nucleic acid set may be comprised within a vector or a set of vectors, for example, an expression vector or a set of expression vectors. In specific examples, the vector or set of vectors comprises one or more viral vectors, e.g., a lentiviral vector or retroviral vector.

In some embodiments, at least about 5×10⁴ anti-GPC3−CAR T cells per kg are administered to the patient. In some examples, about 5×10⁴ to about 1×10¹² anti-GPC3-CAR T cells/kg are administered to the patient.

In some embodiments, the therapeutic agent may comprise an anti-GPC3 targeted polypeptide or polypeptide fusion, for example, an anti-GPC3 antibody, an anti-GPC3 bi- or multiple specific protein or an anti-GPC3 antibody-drug-conjugate.

In some embodiments, the administration of the anti-GPC3 therapeutic agent is effective in achieving stable disease according to RECIST (e.g., RECIST 1.1) as measured by computerized tomography (CT) scan. Alternatively, or in addition, the administration of the anti-GPC3 therapeutic agent is achieving an objective response according to RECIST (e.g., RECIST 1.1) as measured by computerized tomography (CT) scan.

Any of the methods disclosed herein may further comprise administering at least one immunomodulatory agent to the patient in parallel or sequential to the therapeutic agent. In some embodiments, the immunomodulatory agent can be an immune checkpoint inhibitor or an immunostimulatory cytokine. Alternatively, or in addition, the method may further comprise subjecting the patient to a lymphocyte reduction treatment, which may comprise cyclophosphamide, fludarabine, or a combination thereof.

Also within the scope of the present disclosure are anti-GPC3 therapeutic agents (e.g., those disclosed herein) for use in treating a patient diagnosed with myxoid/round cell liposarcoma, as well as use of any of the anti-GPC3 therapeutic agents disclosed herein for manufacturing a medicament for use in treating a patient diagnosed with myxoid/round cell liposarcoma.

Further, the present disclosure also provides a method for diagnosing a patient having myxoid/round cell liposarcoma or for selecting a patient for treatment of the disease. The method may comprise:
(a) obtaining a tissue section from a tumor biopsy sample obtained from a patient candidate, the section having a thickness between 3 μm and 15 μm,
(b) immunostaining preferably by IHC with an antibody that specifically binds to GPC3, more specifically using the antibody GC33,
(c) determining the cytoplasmic/membranous H-score, and
(d) diagnosing the patient candidate as having or suspected of having myxoid/round cell liposarcoma based on the H-score.

In some instances, an H-score of greater than 30 is indicative of disease occurrence. In some instances, a patient having an H-score greater than 30 can be selected for treatment.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1. shows images of an FFPE tissue section of Myxoid/round cell liposarcoma stained by IHC with the antibody GC33, with the box indicating a particular field viewed at A. 1×, B. 4×, and C. 20× magnification. Cells within each field of view were scored as 1+ for weak staining, 2+ for moderate staining and 3+ for strong staining; examples of 1+, 2+ and 3+ are boxed in C.

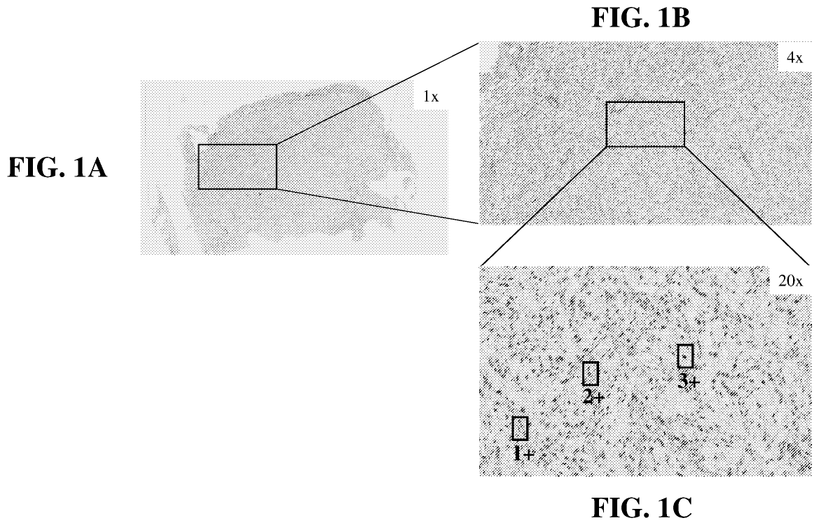

FIG. 2. shows images of IHC staining by the antibody GC33 in healthy FFPE tissue sections of A. Breast), B. heart, C. stomach and, D. kidney at 20× magnification.

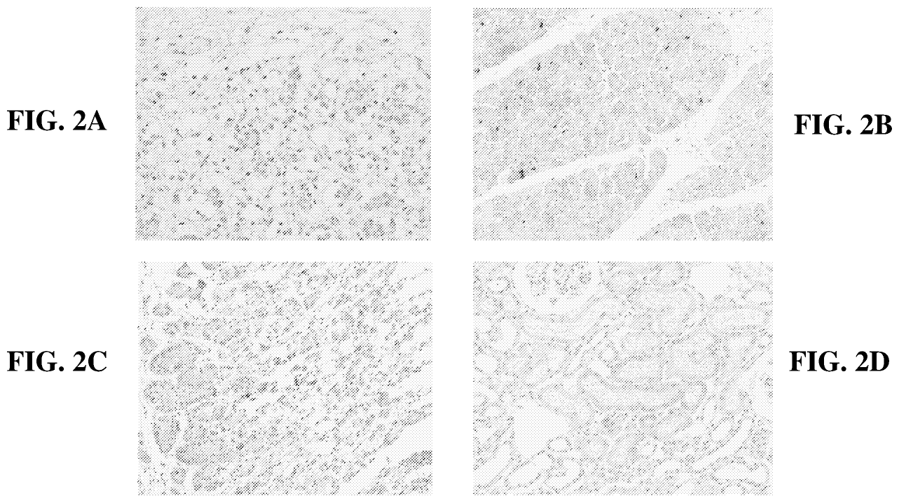

FIG. 3. shows images of IHC staining by the antibody GC33 in various cancer types namely in FFPE tissue sections of A. smooth muscle (negative control; H-score—0), B. hepatocellular carcinoma (H-score—280), C. non-small cell lung carcinoma (H-score—260), D. Merkel cell carcinoma (H-score—130), and E. liposarcoma (H-score—120) at 20× magnification. All staining fields show both membranous and cytoplasmic GPC3 staining.

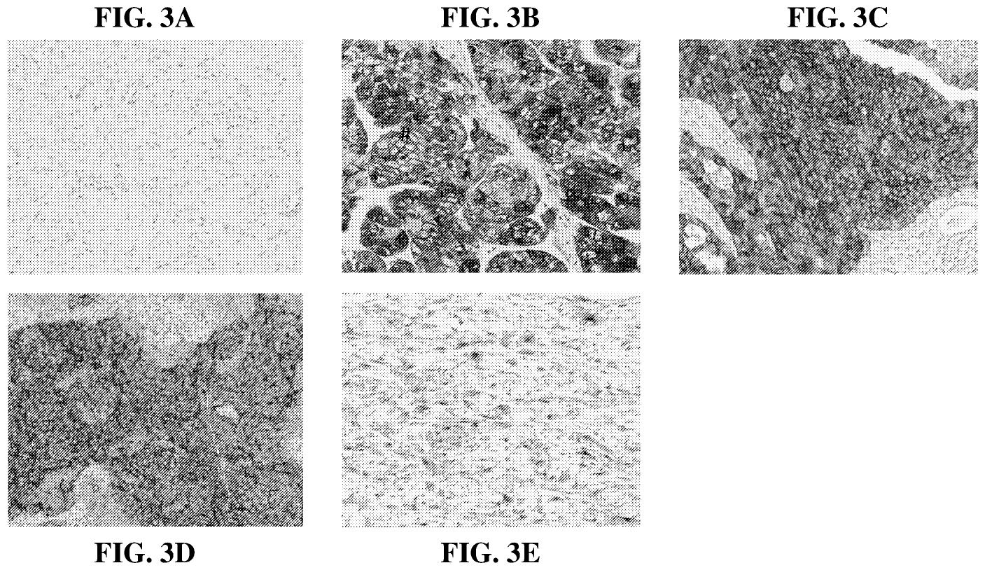

FIG. 4. shows images of IHC staining by the antibody GC33 in liposarcoma subtypes namely in FFPE tissue sections of A. myxoid/round cell liposarcoma (H-score—140), B. pleiomorphic liposarcoma (H-score—0), C. well differentiated liposarcoma (H-score—0), and D. mixed liposarcoma (H-score—0), at 20× magnification.

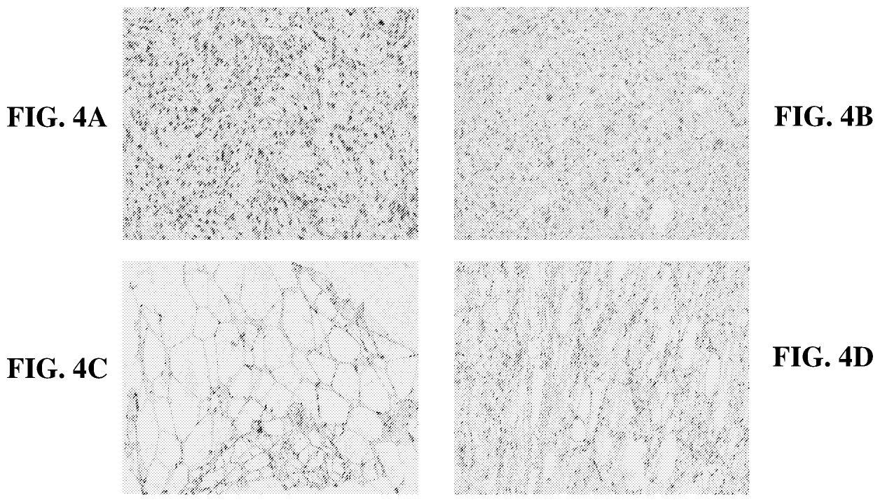

### DETAILED DESCRIPTION OF DISCLOSURE

### Abbreviations

Throughout the detailed description and examples of the invention the following abbreviations are used:
CAR Chimeric antigen receptor
CDR Complementarity determining region in the immunoglobulin variable regions, defined using the Kabat numbering system, unless otherwise indicated
FFPE Formalin-fixed, paraffin-embedded
FR Antibody framework region: the immunoglobulin variable regions excluding the CDR regions
GOT Glutamate Oxaloacetate Transaminase
GPC Glypican
HCC Hepatocellular carcinoma
Hrs Hours
HSC Hematopoietic stem cells
HRP Horseradish peroxidase
IgG Immunoglobulin G
IF Immunofluorescence
IHC Immunohistochemistry
ISH In situ hybridization
LPS Liposarcoma
mAb or Mab or MAb Monoclonal antibody
mins Minutes
MRCLS Myxoid/Round Cell Liposarcoma
NSCLC Non-small cell lung cancer
PCR Polymerase chain reaction
RT room temperature
V region The segment of IgG chains which is variable in sequence between different antibodies. It extends to Kabat residue 109 in the light chain and 113 in the heavy chain.
VH Immunoglobulin heavy chain variable region
VL Immunoglobulin light chain variable region
VK Immunoglobulin kappa light chain variable region

### Definitions

Terms “administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. As used herein, “treatment” refers to clinical intervention in an attempt to alter the natural course of the individual or cell being treated and can be performed before or during the course of clinical pathology. Desirable effects of treatment include preventing the occurrence or recurrence of a disease or a condition or symptom thereof, delaying onset of the disease or condition, alleviating a condition or symptom of the disease, diminishing any direct or indirect pathological consequences of the disease, decreasing the rate of disease progression, ameliorating, or palliating the disease state, and achieving remission or improved prognosis.

Terms “antibodies” or “antibody”, also called “immunoglobulins” (Ig), generally comprise four polypeptide chains, two heavy (H) chains and two light (L) chains, and are therefore multimeric proteins, or comprise an equivalent Ig homologue thereof (e.g., a camelid antibody comprising only a heavy chain, single-domain antibodies (sdAb) or nanobody which can be either be derived from a heavy or light chain). The term “antibodies” includes antibody-based binding protein, modified antibody format retaining target binding capacity. The term “antibodies” also includes full length functional mutants, variants, or derivatives thereof (including, but not limited to, murine, chimeric, humanized and fully human antibodies) which retain the essential epitope binding features of an Ig molecule, and includes dual specific, bispecific, multispecific, and dual variable domain Igs. Ig molecules can be of any class (e.g., IgG, IgE, IgM, IgD, IgA, and IgY), or subclass (e.g., IgG1, IgG2, IgG3, IgG4, IgA1, and IgA2) and allotype. Ig molecules may also be mutated, e.g., to enhance or reduce affinity for Fcγ receptors or the neonatal Fc receptor (FcRn).

The term “antibody fragment”, as used herein, relates to a molecule comprising at least one polypeptide chain derived from an antibody that is not full length and exhibits target binding. Antibody fragments are capable of binding to the same epitope or target as their corresponding full-length antibody. Antibody fragments include, but are not limited to (i) a Fab fragment, which is a monovalent fragment consisting of the variable light (VL), variable heavy (VH), constant light (CL) and constant heavy 1 (CHi) domains; (ii) a F(ab′)₂ fragment, which is a bivalent fragment comprising two Fab fragments linked by a disulfide bridge at the hinge region (reduction of a F(ab′)₂ fragment result in two Fab′ fragment with a free sulfhydryl group); (iii) a heavy chain portion of a Fab (Fa) fragment, which consists of the VH and CHi domains; (iv) a variable fragment (Fv) fragment, which consists of the VL and VH domains of a single arm of an antibody; (v) a domain antibody (dAb) fragment, which comprises a single variable domain; (vi) an isolated complementarity determining region (CDR); (vii) a single chain Fv fragment (scFv); (viii) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites; (ix) a linear antibody, which comprises a pair of tandem Fv segments (VH-CH1-VH-CH1) which, together with complementarity light chain polypeptides, form a pair of antigen binding regions; (x) Dual-Variable Domain Immunoglobulin; (xi) other non-full length portions of immunoglobulin heavy and/or light chains, or mutants, variants, or derivatives thereof, alone or in any combination.

The term “antibody-based binding protein”, as used herein, may represent any protein that contains at least one antibody-derived VH, VL, or CH immunoglobulin domain in the context of other non-immunoglobulin, or non-antibody derived components. Such antibody-based proteins include, but are not limited to (i) Fc-fusion proteins of binding proteins, including receptors or receptor components with all or parts of the immunoglobulin CH domains, (ii) binding proteins, in which VH and or VL domains are coupled to alternative molecular scaffolds, or (iii) molecules, in which immunoglobulin VH, and/or VL, and/or CH domains are combined and/or assembled in a fashion not normally found in naturally occurring antibodies or antibody fragments.

The term “antibody-Drug conjugate” or “ADC” refers to an antibody or antibody fragment to which toxins (or drugs) have been linked. In an ADC, toxins are conjugated to the antibody or antibody fragment by cleavable or non-cleavable linkers.

The term “anti-GPC3 therapeutic agent” refers to a therapeutic agent that is targeted to GPC3 The desired or beneficial effects can include: (a) the inhibition of the further growth or diffusion of cancer cells; (b) the killing of cancer cells; (c) the inhibition of cancer recurrence; (d) the alleviation, reduction, mitigation, or inhibition of cancer-related symptoms (pain, etc.) or reduction in the frequency of the symptoms; and (e) improvement in the survival rate of the patient. Targeted therapeutic agents comprise a binding moiety that specifically binds to the 10 GPC3 antigen expressed on tumor cells. Some non-limiting examples of anti-GPC3 therapeutic agents includes genetically modified cells with chimeric antigen receptor polypeptides, anti-GPC3 antibodies and/or antibody-drug conjugates.

The term “aiding diagnosis” is used herein to refer to methods that assist in making a clinical determination regarding the presence, degree, or other nature, of a particular type of symptom or condition of cancer, such as LPS or non-LPS. Diagnosis of cancer, such as LPS or its subtypes such as MRCLS, may be made according to any protocol that one of skill of art would use. The term “brightfield type image” or “virtual stained image” (VSI) refers to an image of abiological sample that simulates that of an image obtained from a brightfield staining protocol. The image has similar contrast, intensity, and coloring as a brightfield image. This allows features within a biological sample, including but not limited to nuclei, epithelia, stroma or any type of extracellular matrix material features, to be characterized as if the brightfield staining protocol was used directly on the biological sample.

The terms “cancer”, “cancerous”, “tumor” or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, gastrointestinal (tract) cancer, renal cancer, ovarian cancer, liver cancer, lymphoblastic leukemia, lymphocytic leukemia, prostate cancer, thyroid cancer, melanoma, pancreatic cancer, glioblastoma multiforme, stomach cancer, bladder cancer and sarcoma.

The term “chemical agent” may include one or more chemicals capable of modifying the fluorophore or the cleavable linker (if present) between the fluorophore and the binder. A chemical agent may be contacted with the fluorophore in the form of a solid, a solution, a gel, or a suspension. Suitable chemical agents useful to modify the signal include agents that modify pH (for example, acids or bases), electron donors (e.g., nucleophiles), electron acceptors (e.g., electrophiles), oxidizing agents, reducing agents, or combinations thereof.

The term “chimeric antigen receptor” or “CAR” refers to an artificial antigen receptor that is engineered to be expressed on an immune effector cell and specifically bind a cell-surface antigen and employs one or more signaling molecules to activate such immune effector cell. In case the immune effector cell is a T cell, activation may lead to cell killing, proliferation and/or cytokine production (Jena et al., 2010) CARs may be used as a therapy with adoptive cell transfer. Hematopoietic cells, e.g., PBMCs, are removed from a patient and modified so that they express the CAR. The CAR may be expressed with specificity to a tumor associated antigen mediated by an extracellular antigen-binding domain, e.g., an scFv and recognition is independent of a human leukocyte antigen (HLA) presentation, or an engineered T cell receptor which still recognizes HLA-presented peptides (Zhang & Wang, 2019). Preferred within this invention are CARs in the narrow sense with an antigen-binding domain, e.g., a single chain variable fragment (scFv), capable of binding a tumor-associated antigen independent of HLA presentation. CARs further comprise an intracellular activation domain, a transmembrane domain, and optionally a hinge domain. The specificity of CAR designs may be derived from ligands of receptors (e.g., peptides).

The term “cleavable linker” may be designed to be cleaved extracellularly in the tumor environment or intracellularly within the lysosome Cleavable linkers exploit differential conditions of reducing power or enzymatic degradation that can be present either outside or inside the target cell. In some embodiments of antibody-drug conjugates, the cleavable linkers may be dipeptides (e.g., valine-citrulline and alanine-alanine).

The term “fluorescent marker” refers to a fluorophore that selectively stains particular subcellular compartments. Examples of suitable fluorescent marker (and their target cells, subcellular compartments, or cellular components if applicable) are well known in the art.

The term “fluorophore” refers to a chemical compound, which when excited by exposure to a particular wavelength of light, emits light (at a different wavelength). The terms “fluorescence”, “fluorescent”, or “fluorescent signal” all refer to the emission of light by an excited fluorophore. Fluorophores may be described in terms of their emission profile, or “color.” For example, green fluorophores (for example, Cy3, FITC, and Oregon Green) may be characterized by their emission at wavelengths generally in the range of 515-540 nanometers. Red fluorophores (for example Texas Red, Cy5, and tetramethylrhodamine) may be characterized by their emission at wavelengths generally in the range of 590-690 nanometers. Examples of fluorophores are well known in the art (WO2011138462A1; (Giepmans et al., 2006; Zhang et al., 2002).

The term “binder” refers to a biological molecule that may bind to one or more targets in the biological sample. A binder may specifically bind to a target. Suitable binders may include one or more of natural or modified peptides, proteins (e.g., antibodies, affibodies, or aptamers), nucleic acids (e.g., polynucleotides, DNA, RNA, or aptamers); polysaccharides (e.g., lectins, sugars), lipids, enzymes, enzyme substrates or inhibitors, ligands, receptors, antigens, haptens, and the like. A suitable binder may be selected depending on the sample to be analyzed and the targets available for detection. For example, a target in the sample may include a ligand and the binder may include a receptor or a target may include a receptor and the probe may include a ligand. Similarly, a target may include an antigen and the binder may include an antibody or antibody fragment or vice versa.

The term “in situ” generally refers to an event occurring in the original location, for example, in intact organ or tissue or in a representative segment of an organ or tissue. In some embodiments, in situ analysis of targets may be performed on cells derived from a variety of sources, including an organism, an organ, tissue sample, or a cell culture. In situ analysis provides contextual information that may be lost when the target is removed from its site of origin. Accordingly, in situ analysis of targets describes analysis of target-bound probe located within a whole cell or a tissue sample, whether the cell membrane is fully intact or partially intact where target-bound probe remains within the cell. Furthermore, the methods disclosed herein may be employed to analyze targets in situ in cell or tissue samples that are fixed or unfixed.

The term “diagnosis” is used herein to refer to the identification or classification of a molecular or pathological state, disease or condition. For example, “diagnosis” may refer to identification of a particular type of sarcoma. “Diagnosis” may also refer to the classification of a particular sub-type of LPS.

The term “homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched or homologous when the sequences are optimally aligned then the two sequences are 80% homologous. Generally, the comparison is made when two sequences are aligned to give maximum percent homology. For e.g., the comparison can be performed by a BLAST algorithm wherein the parameters of the algorithm are selected to give the largest match between the respective sequences over the entire length of the respective reference sequences.

The term “monoclonal antibody”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modified “monoclonal” indicates the character of the antibody as being obtained from a substantially homogeneous population of antibodies and is not to be construed as requiring production of the antibody by any particular method.

Terms “multispecific”, “multispecific antigen-binding” and/or “multispecific molecules” are interchangeable. They comprise a first antigen binding domain, and a second antigen-binding domain each of which bind different molecules each referred to as the target molecule. The target molecule may be an internalizing effector protein. As used herein, the expression “simultaneous binding,” in the context of a multispecific antigen-binding molecule, means that the multispecific antigen-binding molecule is capable of contacting both a target molecule (T) and an internalizing effector protein (E) for at least some period of time under physiologically relevant conditions to facilitate the physical linkage between T and E. Binding of the multispecific antigen-binding molecule to the T and E components may be sequential; e.g., the multispecific EP3,722,318 A1 antigen-binding molecule may first bind T and then bind E, or it may first bind E first and then bind T. In any event, so long as T and E are both bound by the multispecific antigen-binding molecule for some period of time (regardless of the sequential order of binding), the multispecific antigen-binding molecule will be deemed to “simultaneously bind” T and E for purposes of the present disclosure. Without being bound by theory, the enhanced inactivation of T is believed to be caused by the internalization and degradative rerouting of T within a cell due to its physical linkage to E. The multispecific antigen-binding molecules of the present invention are thus useful for inactivating and/or reducing the activity and/or extracellular concentration of a target molecule without directly blocking or antagonizing the function of the target molecule. The multispecific molecule can be a single multifunctional polypeptide, or it can be a multimeric complex of two or more polypeptides that are covalently or non-covalently associated with one another. Any of the multispecific molecules or variants thereof, may be constructed using standard molecular biological techniques (e.g., recombinant DNA and protein expression technology), as will be known to a person of ordinary skill in the art.

The term “modified antibody format”, as used herein, encompasses antibody-drug-conjugates (ADCs), polyalkylene oxide-modified scFv, monobodies, diabodies, camelid antibodies, domain antibodies, bi-, tri- or multispecific antibodies, IgA, or two IgG structures joined by a J chain and a secretory component, shark antibodies, new world primate framework and non-new world primate CDR, IgG4 antibodies with hinge region removed, IgG with two additional binding sites engineered into the CH3 domains, antibodies with altered Fc region to enhance or reduce affinity for Fc gamma receptors, dimerized constructs comprising CH3, VL, and VH, and the like.

The term “non-cleavable linkers” refers to linkers that require the ADC to be internalized, the antibody-linker component needs to be degraded by lysosomal proteases for the toxins to be released. Conjugation of the linker to the antibody may also vary. Conjugation may rely on the presence of lysine and cysteine residues within the polypeptide structure of the antibody as the point of conjugation. Reactive groups on the linker can e.g. be conjugated to the side chain of lysine residues through amide or amidine bond formation. Conjugation via cysteine residues requires a partial reduction of the antibody. Alternatively, site-specific enzymatic conjugation can be used. This requires enzymes that react with the antibody and can induce site- or amino acid sequence-specific modifications. Peptide sequences recognized by these enzymes may have to be inserted into the genetically engineered antibodies or fragments to be conjugated. Enzymes which have been used for such purpose are sortase, transglutaminase, galactosyltransferase, sialyltransferase and tubulin-tyrosine ligase. An overview of ADC linker conjugation and toxins can be found in (Ponziani et al., 2020). An overview of conjugation of toxins to antibody fragments can be found in (Aguiar et al., 2018). The type of linker and the method of conjugation used to conjugate the toxin to the antibody or antibody fragment may determine the drug-to-antibody ratio (DAR).

The term “oligonucleotide,” as used herein, refers to short, single stranded polynucleotides that are at least about seven nucleotides in length and less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “pharmaceutically acceptable” refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions. Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants.

The term “polynucleotide” or “nucleic acid,” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component as described in the art (for e.g. see WO 2013/148448).

The term “primary anti-GPC3 antibody” refers to an antibody that binds specifically to GPC3, e.g., GC33, in a tissue section, and is generally the first antibody used in an immunostaining assay of GPC3 expression, e.g., immunohistochemistry and immunofluorescence in a tumor sample.

The term “sample”, as used herein, refers to a composition that is obtained or derived from a patient that contains a cellular and/or other molecular entity that is to be characterized and/or identified, for example based on physical, biochemical, chemical and/or physiological characteristics.

The term “secondary antibody” refers to an antibody that binds specifically to a primary anti-GPC3 antibody, thereby forming a bridge between the primary antibody and a subsequent detection reagent, if any, in an immunostaining assay of GPC3 expression, e.g., IHC and IF or in situ hybridization.

The term “subject” includes any organism, preferably an animal, more preferably a mammal (e.g., rat, mouse, cynomolgus monkey and human). “Patient” or “subject” refers to any single subject for which therapy is desired or that is participating in a clinical trial, epidemiological study or used as a control, including humans and mammalian veterinary patients such as mouse, rat & cynomolgus monkey. As used herein, the term “patient” refers to a human or non-human animal. Typically, the terms “subject”, “individual”, and “patient” may be used interchangeably herein in reference to a subject. As such, a “patient” includes a human or non-human mammal that is being treated and/or diagnosed for/with a disease, such as cancer.

The term “tissue sample” is meant a collection of similar cells obtained from a tissue of a subject. The source of the tissue sample may be solid tissue as from a fresh, frozen and/or preserved tissue sample. The tissue sample may also be primary or cultured cells or cell lines taken from and/or derived from an individual. The tissue sample may contain compounds which are not naturally intermixed with the tissue in nature such as preservatives, anticoagulants, buffers, fixatives, nutrients, antibiotics, or the like. The tissue sample may also be a fluid isolated from a subject. In a non-limiting aspect, examples of such samples include plasma, serum, spinal fluid, lymph, whole blood or any blood fraction, blood derivatives, blood cells, tumor, any sample obtained by lavage (e.g., samples derived from the bronchi), and samples of components constituting cell cultures in vitro.

The term “therapeutic agent” is a chemical compound or biological molecule useful in the treatment of cancer. Classes of therapeutic agents include, but are not limited to: alkylating agents, antimetabolites, kinase inhibitors, spindle poison plant alkaloids, cytotoxic/antitumor antibiotics, topoisomerase inhibitors, photosensitizers, and antibodies and fusion proteins that block ligand/receptor signaling in any biological pathway that supports tumor maintenance and/or growth. Therapeutic agents useful in the treatment methods of the present invention include cytostatic agents, cytotoxic agents, antibody-drug conjugates, chimeric antigen receptors polypeptides and immunotherapeutic agents.

The term “therapeutically effective amount” refers to an amount of a therapeutic agent effective to “treat” a cancer in a subject or mammal by achieving at least one positive therapeutic effect, such as for example, reduced number of cancer cells, reduced tumor size, reduced rate of cancer cell infiltration into peripheral organs, and reduced rate of tumor metastasis or tumor growth. Positive therapeutic effects in cancer can be measured in a number of ways (See, (Weber, 2009).

The term “tissue section” refers to a single part or piece of a tissue sample, e.g., a thin slice of tissue cut from a sample of a normal tissue or of a tumor.

The term “toxin” refers to a cytotoxic and/or cytostatic agent that can be based on a synthetic, plant, fungal, or bacterial molecule. Cytotoxic or cytostatic means that they inhibit the growth of and/or inhibit the replication of and/or kill cells, particularly malignant cells typically due to their increased turnover.

Terms “treat” or “treating” means to administer a therapeutic agent, such as a composition containing any of the antibodies or antigen binding fragments of the present invention, internally or externally to a subject or patient having one or more disease symptoms, or being suspected of having a disease, for which the agent has therapeutic activity. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom (also referred to as the “therapeutically effective amount”) may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom.

### DETAILED DESCRIPTIONS

Sarcomas are rare group of malignancies of mesenchymal origin accounting for about 20% of all pediatric and about 1% of adult solid tumors (Abaricia & Hirbe, 2018; Hui, 2016). Sarcomas are broadly classified as (i) soft tissue sarcoma (STS) and (ii) sarcomas of the bone. STS has been reported to have an incidence of approximately 3.4 per 100,000 with median age of diagnosis at 59 (as per the surveillance, epidemiology and end results program of the national cancer institute, and the bone sarcoma are even rarer with approximately 0.2% of all cancer diagnosis (Hui, 2016). Liposarcoma (LPS) arise from adipose tissue and is a malignant neoplasm affecting fat differentiation. It is the most common type of STS and represents 17-25% of all newly diagnosed adult sarcomas (Dodd, 2012; Henze & Bauer, 2013; Singhi & Montgomery, 2011). The world health organization (WHO) classifies LPS histologically into four subtypes namely, (i) atypical lipomatous tumor (ALT)/well-differentiated LPS (WDLPS; 40-45% of all LPS; low-grade with 5-year survival rate of 93%), (ii) de-differentiated LPS (DDLPS; high-grade with 5-year survival rate of 45%), (iii) myxoid LPS (MLPS; low grade but 10% of patients develop metastasis with 10-year survival rate of 60%)/round-cell LPS (RCLPS; 30-35% of all LPS; high-grade), together Myxoid/round cell liposarcoma (MRCLS), (iv) pleomorphic LPS (PLPS; <15% of all LPS; high-grade with poor prognosis). A fifth subtype known as the mixed LPS consists of histological combination of one or more subtypes. Both WDLPS and DDLPS are nowadays categorized together as they share same underlying genetic alterations and display similar clinical features (Amer et al., 2020; Henze & Bauer, 2013; Jo & Fletcher, 2014).

Myxoid/round cell liposarcoma (MRCLS) accounts for approximately 30% of LPS and tends to occur in slightly younger age group with peak incidence in the fourth decade of life. These tumors preferentially develop in the lower extremities within the thigh or popliteal space (75%), while they almost never develop in the retroperitoneum. Overall, local recurrence rates reported for MRCLS range from 15% to 30%. Several studies have reported a 20-40% risk of distant metastases. Interestingly, MRCLS has an unusual pattern of metastasis with common metastases to other soft tissue sites, intraabdominal/retroperitoneal spaces or bone (66%) and lower rates of isolated lung metastases (34%) compared to DDL and other STS. Reported disease specific mortality for MRCLS ranges from 12% to 30%. Cytogenic and molecular analyses characterize MRCLS by the recurrent reciprocal translocation t(12;16)(q13;p11) resulting in the FUS-DDIT3 gene fusion in over 95% of cases. Therapies targeted at the inhibition of these fusion proteins are being developed for the treatment of MRCLS (Lee et al., 2018).

Standard treatment for MRCLS include surgical resection for localized, primary disease. In patients with advanced or metastatic disease, MRCLS is known for its sensitivity to radiation therapy and cytotoxic chemotherapy in comparison to the other LPS subtypes. However, despite the appropriate local treatment, about 40% of patients do relapse. Chemotherapy is usually administered for advanced or unresectable disease. Usually, this includes doxorubicin, alone or in combination with ifosfamide, as the first-line therapy and trabectedin as the second-line therapy. Studies carried out on doxorubicin-based regimens in MRCLS showed an overall response rate of 45-50%. Trabectedin proved extremely active in MRCLS. Today, trabectedin is approved as a second-line therapy of STS and plays a key role within the so-called ‘histology-driven’ medical therapy of STS. In MRCLS, trabectedin may obtain a response rate in the 50% range, if assessed through standard dimensional criteria, and a 6-month progression-free survival (PFS) in the 80% range. However, this is true for two drugs: doxorubicin and trabectedin. When the tumour is resistant to these drugs, no other medical option at the moment has an outstanding activity in MRCLS patients (Regina & Hettmer, 2019; Sanfilippo et al., 2013). Tyrosine kinase inhibitors Pazopanib and Suntinib were evaluated but could not warrant their use alone in treatment of MRCLS. Recently, immunotherapy regimes using gene modified T cells for MRCLS patients have begun (for e.g. ClinicalTrials.gov Identifier: NCT03450122 and ClinicalTrial.gov identifier NCT03399448) (Abaricia & Hirbe, 2018; Lee et al., 2018; Regina & Hettmer, 2019; Suarez-Kelly et al., 2019). Still, treatment options for patients with inoperable or metastatic MRCLS continue to be poor and new therapy options are required for this high-medical need indication.

It is reported herein that due to the complexity of staining patterns observed in various tumor tissue sections stained with an anti-GPC3 antibody (GC33), new scoring rules were needed to quantify the level of GPC3 expression accurately and reliably across multiple tissue samples and pathologists doing the scoring. And upon surveying the expression profile of GPC3 in solid tumor biopsies from cancer patients to better understand GPC3 positive tumor prevalence and for indication prioritization for an anti-GPC3 therapy, a sub-group of LPS, namely MRCLS, as a relatively high GPC3 expressing patient population was identified surprisingly, suggesting this indication, i.e., MRCLS, to be prioritized and/or selected for treating with an anti-GPC3 therapeutic agent.

Glypican-3 (GPC3 also called DGSX, GTR2-2, MXR7, OCI-5, SDYS, SGB, SGBS, and SGBS1) is an oncofetal tumor antigen that is an attractive target for anti-GPC3 therapy due to its highly restricted expression on normal tissue and high prevalence in several adult and pediatric solid tumors (Ho & Kim, 2011). GPC3 expression has been observed in a variety of human cancers, e.g., ovarian, renal, colorectal, pancreatic, liver and melanoma. Therefore, an approach to quantifying and identifying sub-population of patients eligible and thereby, benefitting the anti-GPC3 treatment becomes critical for success.

The present disclosure is based on the surprising finding that MRCLS as a niche-subtype of LPS showing an increased positivity of GPC3 expression with high frequency. The frequency of GPC3 expression within this subtype of LPS patients allows for the selection of MRCLS patients with high unmet medical need to be treated with anti-GPC3 therapeutic agents with a high chance for clinical benefit. Accordingly, one aspect of the present disclosure features a method of treating a patient diagnosed with MRCLS, the method comprising administering an anti-Glypican-3 (GPC3) therapeutic agent to the patient. Such patents may be identified by any of the diagnostic methods disclosed herein for detecting presence of GPC3 in tumor tissue samples.

In one embodiment, patient is selected for treatment by diagnosing MRCLS. Liposarcomas have not been described for high GPC3 expression, neither was any subgroup of it. The inventors surprisingly identified MRCLS as a patient group with relatively high GPC3 expression enabling such target GPC therapy to be successful, and using scoring rules based on the GC33 antibody to accurately quantify GPC3 expression reliably across multiple tissue samples in order to find such GPC3 expression in tumors. Due to the relatively high positivity of GPC3 expression in MRCLS patients, a patient stratification for GPC3 expression may not be required in order to achieve substantial response rates with anti-GPC3 therapeutic agents. MRCLS is generally diagnosed by imaging modalities such as CT or MRI followed mainly histological assessment post-hematoxylin & Eosin staining on FFPE tissue section of biopsy samples. Histologically, MRCLS is characterized as a multinodular mass composed of a myxoid matrix of hyaluronic acid and signet ring lipoblasts containing low central cellularity and increased peripherally cellularity of fusiform or round cells with a delicate plexiform capillary vascular network. As these tumors lose their differentiation, they develop areas of increased cellularity. Next, as majority of patients diagnosed with MRCLS carry the reciprocal translocation t(12;16)(q13;p11) resulting in the FUS-DDIT3 (CHOP) and therefore, the diagnosis can be confirmed with high confidence, for example using FISH for DDIT3 (CHOP) on FFPE sections (Fritchie et al., 2012). Another non-limiting example for diagnosing MRCLS patients is by IHC staining for NY-ESO-1 (Hemminger & Iwenofu, 2013).

In the present invention, preferred examples of biological samples used for detecting the expression level of GPC3 in tissues include subject-derived preparations. The subject-derived preparation is preferably a tissue obtained from the subject, more preferably a tissue of the MRCLS patient. The GPC3 expression levels in MRCLS patients may be determined by immunostaining and/or by in situ hybridization. In preferred embodiment, the patient selected for treatment of MRCLS expresses GPC3. In a non-limiting aspect, the present invention may also provide a method for determining the efficacy of anti-GPC3 therapeutic agent or determining the continuation of anti-GPC3 therapeutic agent from the concentration of free GPC3 as well as the expression level of GPC3 detected in tissues by the method described below.

Method of immunostaining and in situ hybridization are well known in the art (see (Lu et al., 2021; Wang et al., 2018; Zhou et al., 2018); WO 2006/006693; WO2009/116659; WO 2013/148448; WO 2014/165422; WO 2014/097648). Either of the diagnostic assays may have common procedural steps.

### I. Identification of MRCLS Patients for Treatment by Anti-GPC3 Therapeutic Agents

In some aspects, the present disclosure features a method for diagnosing myxoid/round cell liposarcoma (MRCLS) patients suitable for treatment by any of the anti-GPC3 therapeutic agents disclosed herein (e.g., anti-GPC3 CAR-T cell therapy). Briefly, tumor biopsy samples may be collected from a candidate patient and examined for presence and/or level of GPC3 in the biopsy samples via, e.g., an immunostaining assay. Tumor biopsy samples showing presence of GPC3 or a certain level of GPC3 may be identified and the patients from whom the biopsy samples are obtained may be identified as suitable patients for treatment by an anti-GPC3 therapy, e.g., those disclosed herein. In some embodiments, fixed tissue samples may be used in the diagnostic assays disclosed herein. Alternatively, fluid samples may be used.

Further, the level of GPC3 may also be used as a biomarker for assessing efficacy of any of the treatment methods as disclosed herein. Accordingly, the present disclosure also provides a method for assessing treatment efficacy of MRCLS patients who is receiving or will be receiving a treatment for MRCLS, for example, any of the anti-GPC3 therapy as disclosed herein. The level of GPC3 in a suitable biological sample from a MRCLS patient may be measured using any of the assay methods disclosed here or those known in the art. The efficacy of GPC3-targeting drug therapy for the MRCLS patients as disclosed herein may be determined before the start of anti-GPC3 therapeutic agents of a patient or before the continuation of the anti-GPC3 therapy. For example, a physician may use GPC3 expression score(s) e.g., as disclosed herein, as a guide in deciding how to treat a patient who has been diagnosed with a type MRCLS that is susceptible to treatment with an anti-GPC3 therapeutic agent. In some instances, the physician may use a diagnostic test, with any of the methods disclosed above, to determine GPC3 expression in a tumor tissue sample removed from the patient prior to initiation of treatment with an anti-GPC3 therapeutic agent and/or the other chemotherapeutic agent(s), but it is envisioned that the physician could order the subsequent tests at any time after the individual is administered the first dose of an anti-GPC3 therapeutic agent.

### A. Diagnostic Assays Using Fixed Tissue Samples

(i) Sample Collection and preparation of tissue sections

A tumor biopsy from a MRCLS patient is used to prepare stained tissue sections for scoring GPC3 expression. The biopsy is typically collected from a subject prior to starting treatment with the anti-GPC3 therapeutic agent. Further, biopsies can be obtained during the treatment to confirm GPC3 positivity or even observe upregulation of its expression. Accordingly, tumor samples may be collected from a subject over a period of time. The tumor sample can be obtained by a variety of procedures including, but not limited to, surgical excision, aspiration, or biopsy. In some embodiments, the tissue sample may be first fixed and then dehydrated through an ascending series of alcohols, infiltrated and embedded with paraffin or other sectioning media so that the tissue sample may be sectioned. In an alternative embodiment, a tissue sample may be sectioned and subsequently fixed. In some embodiments, the tissue sample may be embedded and processed in paraffin. Neutral buffered formalin, glutaraldehyde, Bouin's or paraformaldehyde are nonlimiting examples of fixatives. In preferred embodiments, the tissue sample is fixed with formalin. In some embodiments, the fixed tissue sample is also embedded in paraffin to prepare a formalin-fixed and paraffin-embedded (FFPE) tissue sample. Examples of paraffin include, but are not limited to, Paraplast, Broloid and Tissuemay.

It is understood that multiple sections of a single tissue sample may be prepared and analyzed in accordance with the present invention. Each tissue section has a thickness between 3 μm-15 μm, preferably 3 μm-8 μm. In one embodiment, a tissue section was obtained from a tumor biopsy sample, the section having a thickness between 3 μm and 15 μm. In one embodiment, FFPE-fixed tissue section of 5 μm thickness was used in an IHC assay of GPC3 expression in a tumor sample. In another embodiment, FFPE-fixed tissue section of 6 μm thickness was used in an ISH assay of GPC3 expression in a tumor sample. In some embodiments, the scoring process of the invention is performed on FFPE tissue sections of about 3 μm-8 μm, and preferably 5 μm, which are mounted and dried on a microscope slide.

### (ii) Anti-GPC3 Antibodies Used in Immunostaining

As used herein, the primary antibody is an anti-GPC3 antibody (mouse monoclonal Ab GC33; Cat. No. 790-4564; Ventana) used in immunostaining both in IHC and IF. GC33 antibody is specifically directed against the heparan sulfate proteoglycan GPC3. Anti-GPC3 antibody exhibits preferential binding to human GPC3 as compared to other antigens, but this specificity does not require absolute binding specificity. An anti-hGPC3 antibody is considered specific for human GPC3 if its binding is determinative of the presence of human GPC3 in a sample, e.g. without producing undesired results such as false positives in an IHC diagnostic assay. Antibodies, or binding fragments thereof, useful as a primary antibody in the processes and methods of the present invention will bind to human GPC3 with an affinity that is at least two-fold greater, preferably at least ten times greater, more preferably at least 20-times greater, and most preferably at least 100-times greater than the affinity with any non-GPC3 protein. Tissue sections of tumor samples from human subjects may be scored for GPC3 expression using any anti-hGPC3 Ab that produces essentially the same staining results on an FFPE or frozen tissue section of a tumor sample from a human as produced by the GC33 Ab.

Typically, an anti-GPC3 Ab or antigen binding fragment useful in scoring expression of human GPC3 by IHC assay, will exhibit the same degree of specificity for human GPC3 as the GC33 antibody and retain at least 80%, 85%, 90%, 95% or 100% of its human GPC3 binding affinity when that affinity is expressed on a molar basis. It is also intended that an anti-GPC3 antibody or antigen binding fragment useful in the invention can include conservative or non-conservative amino acid substitutions from the GC33 Ab or GC33 that do not substantially alter its binding specificity or affinity.

(iii) Diagnostic Testing for GPC3 Expression by Immunostaining

The present invention identified the niche sub-type MRCLS within the population of LPS with enhanced GPC3 expression. It further provides a process for scoring GPC3 expression in MRCLS tumor tissue sections that have been immunostained with an anti-GPC3 antibody in an IHC or IF assay. In one embodiment, a patient diagnosed with MRCLS is selected for treatment by immunostaining preferably by IHC with an antibody that specifically binds to GPC3, more specifically using the antibody GC33. The results of these scoring processes may be used to select patients for treatment with an anti-GPC3 therapeutic agent.

An IHC or IF assay typically begins with antigen retrieval, which may vary in terms of reagents and methods. Examples of antigen retrieval process are well known in the art (see, e.g., (Leong, 1996). In some embodiments, protease treatment is used for antigen retrieval. In preferred embodiments, paraffin-embedded (FFPE) tissue sections are subjected to heat-induced antigen retrieval process. Both IHC or IF may be used in a direct or an indirect assay. In a direct IHC or IF assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody.

In one embodiment, primary anti-GPC3 antibody that binds specifically to GPC3 preferably in a tissue section is GC33. It is generally the first antibody used in an immunostaining assay of GPC3 expression, e.g., IHC and IF in a tumor sample. In one embodiment, the primary antibody is the only antibody used in the IHC assay. Where the secondary antibody is conjugated to an enzymatic label, a chromogenic or fluorogenic substrate is added to provide visualization of the antigen. Signal amplification occurs because several secondary antibodies may react with either a different or the same epitope on the primary antibody. The secondary antibody binds specifically to a primary anti-GPC3 antibody, thereby forming a bridge between the primary antibody and a subsequent detection reagent, if any, in an immunostaining assay of GPC3 expression, e.g., IHC and IF or in situ hybridization. In one embodiment, the secondary antibody is generally the second antibody used in an IHC assay of GPC3 expression in a tumor sample.

The primary and/or secondary antibody used for IHC or IF typically, will be labeled with a detectable moiety. In some embodiments, the primary antibody is linked to a detectable label, such as paramagnetic ions, radioactive isotopes, fluorochromes, and NM-detectable substances, and the slide is evaluated for GPC3 staining using the appropriate imaging apparatus. In other embodiments, immune complexes between GPC3 and the primary antibody may be detected using a second binding agent that is linked to a detectable label. The second binding agent is preferably a secondary antibody, which is applied to the slide at a concentration and for a period of time sufficient to allow the formation of secondary immune complexes. The slide is then typically washed to remove any non-specifically bound secondary antibody, and the label in the secondary immune complexes is detected. The secondary antibody may be labeled using avidin, streptavidin or biotin, which is independently labeled with a detectable moiety, such as a fluorescent dye (stain), a luminescent dye or a non-fluorescent dye. Numerous labels are available which can be generally grouped into the following categories; (a) Radioisotopes, (b) Colloidal gold particles and, (c) Fluorescent or chemiluminescent labels. Examples of detectable moieties have been extensively disclosed in WO 2013/148448. Some examples include, but not limited to, fluorescein and its derivatives, rhodamine and its derivatives, phycoerythrin, phycocyanin, or commercially available fluorophores such SPECTRUM ORANGE® and SPECTRUM GREEN® and/or derivatives of any one or more of the above.

Various enzyme-substrate labels are available, and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be assessed under a bright-field microscope. In one embodiment, the GPC3 expression in MRCLS was assessed in an IHC chromogenic assay under a bright-field microscope preferably a scanner (Lecia or Ventana, e.g., Ventana DP 200 scanner, Aperio AT2). In another embodiment, the GPC3 expression in MRCLS was assessed in an IF assay under a florescent microscope preferably florescent scanner. Examples of florescent microscopes and are not limited to, inverted, compound, stereo, polarizing preferably confocal microscopes (Leica) and scanner-type (Leica and Ventana, e.g., Ventana DP 200 scanner).

Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Non-limiting examples of enzymatic labels include luciferases (e.g. firefly luciferase and bacterial luciferase; U.S. Pat. No. 4,737,456; WO2013095896A1), luciferin, peroxidase such as horseradish peroxidase (HRP), alkaline phosphatase, β-galactosidase, lactoperoxidase, microperoxidase, and the like. In some embodiments, the label is indirectly conjugated with the antibody. The skilled artisan will be aware of various techniques for achieving this. For example, the antibody can be conjugated with biotin and any of the four broad categories of labels mentioned above can be conjugated with avidin, or vice versa.

Techniques for conjugating enzymes to antibodies are described in (O'Sullivan & Marks, 1981).

Numerous enzyme-substrate combinations are available to those skilled in the art. For a general review of these, see U.S. Pat. Nos. 4,275,149 and 4,318,980. Examples of enzyme-substrate combinations are:
(i) Horseradish peroxidase (HRP) with hydrogen peroxidase as a substrate, wherein the hydrogen peroxidase oxidizes a dye precursor, such as, e.g., 3,3′ diamino benzidine (DAB), which produces a brown end product; 3-amino-9-ethylcarbazole (AEC), which upon oxidation forms a rose-red end product; 4-chloro-1-napthol (CN), which precipitates as a blue end product; and p-Phenylenediamine dihydrochloride/pyrocatecol, which generates a blue-black product; orthophenylene diamine (OPD) and 3,3′,5,5′-tetramethyl benzidine hydrochloride (TMB);
(ii) alkaline phosphatase (AP) and para-Nitrophenyl phosphate, naphthol AS-MX phosphate, Fast Red TR and Fast Blue BB, napthol AS-BI phosphate, napthol AS-TR phosphate, 5-bromo-4-chloro-3-indoxyl phosphate (BCIP), Fast Red LB, Fast Garnet GBC, Nitro Blue Tetrazolium (NBT), and iodonitrotetrazolium violet (INT); and
(iii) β-D-galactosidase (β-D-Gal) with a chromogenic substrate (e.g., p-nitrophenyl-P-D-galactosidase) or fluorogenic substrate (e.g., 4-methylumbelliferyl-P-D-galactosidase).

Any method known in the art for conjugating the antibody molecules to the various moieties may be employed, including those methods described by (David & Reisfeld, 1974; Nygren, 1982; Pain & Surolia, 1981).

### (iv) Immunostaining Scoring Process

After completing the staining process on tissue section of MRCLS patients, the slide is analyzed for GPC3 staining, either by a human, e.g., a pathologist, or a computer programmed to distinguish between specific and non-specific staining results. The analysis may be performed directly by viewing the slide through a microscope at low, medium (e.g. 10-20×) and high power (e.g. 40-63×), or by viewing high resolution images of the slide taken at low, medium and high power. Low and medium power is typically used to detect and for a general overview of stained tumor cells. Medium and high power is typically used to examine individual tumor cells to estimate the number and intensity of viable cells that exhibit at least partial localization of GPC3 to the cell membrane (apical and circumferential) and cytoplasmic staining. Canalicular staining pattern was also recorded. In a preferred embodiment, each stained tissue section in an IHC assayed is assigned an H-score. The H-score comprises (i) estimating, across all of the viable GPC3-stained tumor cells in all of the examined sections, four separate percentages for cells that have no staining, weak staining (+1), moderate staining (+2) and strong staining (+3), wherein a cell must have at least partial membrane and/or cytoplasmic staining to be included in the weak, moderate or strong staining percentages, and wherein the sum of all four percentages equals 100; and (ii) inputting the estimated percentages into the formula of 1*(percentage of tumor cells with 1+ staining intensity)+2*(percentage of tumor cells with 2+ staining intensity)+3*(percentage of tumor cells with 1+ staining intensity), and assigning the result of the formula to the tissue section as the H-score. The H-score combined components of staining intensity with the percentage of positive cells having a range between 0-500, preferably between 0-300. A cut-off of total (cytoplasmic and membranous) H-score >30 in tumor cells is used to determine the positive/negative status of the specimens stained with the GPC3 (GC33) IHC assay.

Such staining procedure can be used to select patients due to the presence of the target antigen GPC3 for the treatment with a GPC3 therapeutic agent. In one embodiment, the MRCLS patients are selected by determining the cytoplasmic/membranous H-score. In some embodiments, the prespecified threshold for GPC3 expression in MRCLS tissue samples is between 30-300. Therefore, in a preferred embodiment, the method of treating a patient diagnosed with the MRCLS is selected by immunostaining, preferably by immunohistochemistry (IHC) staining, preferably with a cytoplasmic/membranous H-score of greater than 30. In another embodiment, the MRCLS patient are selected with an H-score of greater than 30 for the treatment.

In some embodiments, the individuals involved with preparing and analyzing the tissue section by IHC assay do not know the identity of the subject whose sample is being tested; i.e., the sample received by the laboratory is made anonymous in some manner before being sent to the laboratory. For example, the sample may be merely identified by a number or some other code (a “sample ID”) and the results of the IHC assay is reported to the party ordering the test using the sample ID. In preferred embodiments, the link between the identity of a subject and the subject's tissue sample is known only to the individual or to the individual's physician.

In some embodiments, after the test results have been obtained, the diagnostic laboratory generates a test report, which may include any one or more of the following results: the tissue sample was positive or negative for GPC3 expression based on the threshold H-score. The test report may also include guidance on how to interpret the results for predicting if a subject is likely to respond to an anti-GPC3 therapeutic agent. For example, in one embodiment, the patient's tumor is from a MRCLS and if the H-score is at or above a cut-off threshold, the test report may indicate that the patient has a GPC3 expression score that is correlated with response or better response to treatment with the anti-GPC3 therapeutic agent, while if the H-score is below the cut-off threshold, then the test report indicates that the patient has a GPC3 expression score that may correlate with no response or poor response to treatment with anti-GPC3 therapeutic agent. In a preferred embodiment, the method of treating a patient diagnosed with MRCLS wherein the selection comprises immunostaining, preferably immunohistochemical staining of GPC3 in a tumor sample from the patient, wherein the GPC3 expression level is determined and compared to a predetermined threshold level of GPC3 expression, and wherein the patient is selected for treatment in case the patient has a GPC3 expression level equal or higher to the predetermined threshold level.

### (v) Diagnostic Testing for GPC3 Expression by In Situ Hybridization

Another approach to assess the GPC3 expression in MRCLS either supplementing or additionally complimenting the immunostaining assay is by in situ hybridization assay. Sample specimen collection and tissue section preparation are like as disclosed above in case of immunostaining assays. The present invention includes embodiments that relate generally to methods applicable in analytical, diagnostic, or prognostic applications which individually and/or combine chromogenic or immunofluorescence detection with chromogenic or fluorescence based nucleic acid analysis. The disclosed methods relate generally to detection and correlation of different kinds of targets (i.e., protein and/or nucleic acid) from a single biological sample. In some embodiments, methods of detecting multiple targets of the same kind (i.e., protein and/or nucleic acid, respectively) using the same detection channel are disclosed. In such embodiments, correlations can be drawn among the multiple, different kinds of targets.

In one embodiment, a target may include a nucleic acid and the binder may include a complementary nucleic acid. In some embodiments, both the target and the binder may include proteins capable of binding to each other. In some embodiments, the method of detecting multiple targets in a biological sample includes sequential detection of targets in the biological sample. The method generally includes the steps of detecting a first target in the biological sample, optionally modifying the signal from the first target, and detecting a second target in the biological sample. The method may further include repeating the step of modification of signal from the first or second target followed by detecting a different target in the biological sample, and so forth. Detailed method of design & performing in situ hybridization assay is well known in the art. It may be either used with one long stretch of oligonucleotides or may comprise of stretches of short oligonucleotides as probe (see WO 2013/148448; (Wang et al., 2012)). Overall, the assay consists of:
(a) Developing the target nucleic acid sequence—which is a sequence of interest contained in a nucleic acid molecule (e.g., GPC3) in the biological sample. The nucleic acid molecule may be present in the nuclei of the cells of the biological sample (for example, chromosomal DNA) or present in the cytoplasm (for example, mRNA). In some embodiments, a nucleic acid molecule may not be inherently present on the surface of a biological sample and the biological sample may have to be processed to make the nucleic acid molecule accessible by a probe. In some embodiments, the analysis may provide information about the GPC3 gene expression level in the biological sample. In certain embodiments, the target nucleic acid sequence includes a sequence that is part of the gene sequence encoding GPC3. In other embodiments, the target nucleic acid sequence does not include a sequence that is part of the gene sequence which encodes GPC3. Thus, the target nucleic acid sequence may include a sequence that is part of the gene sequence which encodes a different protein than the target protein.
(b) Probes-used to detect the target nucleic acid sequences defined above. It is desirable that the probe binds specifically to the region of the nucleic acid molecule that contains the sequence of interest, e.g., GPC3. Thus, in some embodiments, the probe is GPC3 sequence specific. A sequence-specific probe may include a nucleic acid and the probe may be capable of recognizing a particular linear arrangement of nucleotides or derivatives thereof. In some embodiments, the linear arrangement may include contiguous nucleotides or derivatives thereof that may each bind to a corresponding complementary nucleotide in the probe. In an alternate embodiment, the sequence may not be contiguous as there may be one, two, or more nucleotides that may not have corresponding complementary residues on the probe. Suitable examples of probes may include but are not limited to DNA or RNA oligonucleotides or polynucleotides, peptide nucleic acid (PNA) sequences, locked nucleic acid (LNA) sequences, or aptamers. In some embodiments, suitable probes may include nucleic acid analogs, such as dioxygenin dCTP, biotin dCTP 7-azaguanosine, azidothymidine, inosine, or uridine. In some embodiments, the probe may comprise a nucleic acid probe, a peptide nucleic acid probe, a locked nucleic acid probe or mRNA probe.

The length of the probe may also determine the specificity of binding. In some embodiments, hybridization of smaller probes may be more specific than the hybridization of longer probes, as the longer probes may be more amenable to mismatches and may continue to bind to the nucleic acid depending on the conditions. The probes may further consist of additional nucleic acid sequences (e.g., spacer, head and/or tail sequences). In some embodiments, the probe may have a length in range of from about 4 nucleotides to about 12 nucleotides, from about 12 nucleotides to about 25 nucleotides, from about 25 nucleotides to about 50 nucleotides, from about 50 nucleotides to about 100 nucleotides, from about 100 nucleotides to about 250 nucleotides, from about 250 nucleotides to about 500 nucleotides, or from about 500 nucleotides to about 1000 nucleotides. In some embodiments, the probe may have a length in a range that is greater than about 1000 nucleotides. In one embodiment, the designed GPC3 targeting riboprobes are of length 1-1000 bps preferably 300-700 bps. In another embodiment, the designed GPC3 targeting probes comprise of multiple short aptamers of length 1-100 bps preferably 15-30 bps. In another embodiment, the designed GPC3 targeting probes further consist of additional nucleic acid sequences.

Next, nucleic acid (e.g., mRNA) retrieval process may involve treatment well known in the art (see (Chen et al., 2004; Leong, 1996; Patil et al., 2005; Wang et al., 2012)). In general, indirect assays are used in ISH. In a typical indirect assay, sense or anti-sense nucleic acid may be labelled (for e.g. Digoxigenin and FITC). Unconjugated primary probes and/or aptamers binds to the target nucleic acid sequence. The secondary antibody used for ISH typically will be labeled with a detectable moiety. A labeled secondary antibody binds to the primary probe where the secondary antibody is conjugated to an enzymatic label, a chromogenic or fluorogenic substrate is added to provide visualization of the antigen (e.g., HRP-conjugated anti-DIG). Numerous labels, enzyme substrates, detection by microscope/scanners are available, and examples have been listed & disclosed above within immunostaining. In some embodiments, the label is indirectly conjugated with the antibody. The skilled artisan will be aware of various techniques for achieving this. Signal amplification occurs because several secondary antibodies may react with either different or same epitope in case of on the primary antibody. Alternately, in case of using multiple probes, each of the probe may be conjugated to a different fluorophore or enzyme thereby, enabling primary probe detection within the assay.

In certain embodiments, a biological sample may include an MRCLS tumor tissue sample that may be subjected to ISH using a probe. In some embodiments, an MRCLS tissue sample may be subjected to ISH in addition to immunofluorescence (IF) to obtain desired information regarding the tissue sample. In some embodiments, a probe such a nucleic acid (for example, a DNA) may be directly chemically labeled using appropriate chemistries for the same.

Methods for the detection of nucleic acid sequence such as hybridization are well known. In certain embodiments, a specific nucleic acid sequence is detected by FISH, polymerase chain reaction (PCR) (or a variation of PCR such as in-situ PCR), RCA (rolling circle amplification) or PRINS (primed in situ labeling) (disclosed in detail in WO 2013/148448). In an exemplary embodiment, the specific nucleic acid sequence is detected by FISH. A preferred ISH assay employs the commercially available with RNAscope fluorescent multiplex ACD Biotechnie™ as disclosed in Wang et al. (2012). The target nucleic acid sequence may be analyzed by its presence, absence, expression or amplification level. The protein expression data and the nucleic acid analysis data may be further compared to provide a combined dataset.

### B. Diagnostic Assays Using Fluid Samples

In the present invention, GPC3 expression analysis on MRCLS patients may be performed by various methods on fluid samples. In a non-limiting example, GPC3 concentrations may be measured in serum or plasma isolated in MRCLS patients. In a preferred embodiment, GPC3 concentrations was measured in serum or plasma isolated from patients diagnosed with MRCLS by ELISA. For example, Human Glypican-3 ELISA kit (BioMosaics Inc.) was used to quantify free GPC3 in whole blood serum. Examples of preferred methods for assaying free GPC3 can include immunological methods using an antibody capable of binding to an epitope present in GPC3 and has been disclosed in WO 2006/006693; WO2009/116659; WO 2014/097648; (Hippo et al., 2004).

Further, in another non-limiting example, GPC3 concentrations may be assessed non-invasively in liquid biopsies including circulating tumor DNA (ctDNA) or cell-free DNA (cfDNA) or circulating RNA (ctRNA, e.g., microRNAs), circulating tumor cells (CTC) and/or extracellular vesicles (EVs, e.g., exosomes) (see (Maravelia et al., 2021). In one embodiment, the circulating cells of the MRCLS patient is preferably tumor cells, can be obtained by a non-invasive method. In another embodiment, the circulating cells of the MRCLS patient is isolated based on density, preferably by Ficoll-paque. In another embodiment, the isolated circulating cells, preferably tumor cells, were stained with anti-GPC3 antibody GC33. In a preferred embodiment, the isolated CTCs were stained with anti-GPC3 antibody GC33, preferably by flow cytometry and/or immunostaining. In another embodiment, the CTCs were subjected to in situ hybridization with anti-GPC3 probes. ctDNA or ctRNA may be isolated from circulating cells for e.g. CTCs or directly from the liquid biopsy sample of a MRCLS patient. In an embodiment, the step of confirming the mutation of GPC3 or change in expression (increase or decrease) in a biological sample isolated from a MRCLS patient is preferably by qRT-PCR, next-generation sequencing method, digital PCR, digital droplet PCR etc. However, if it is a general method used for analyzing the sequence of ctDNA or measuring the amount of ctDNA, it is not limited thereto. In another embodiment of the present invention, the method may provide information on MRCLS on the progression of overall disease such as diagnosis, recurrence, advanced stage that helps in deciding further treatment with an anti-GPC3 therapeutic agent. In addition, the present invention may comprise the steps of (a) extracting circulating tumor DNA (ctDNA) from a biological sample of a MRCLS patient cancer to which anti-GPC3 therapeutic agent is administered; (b) administering the anti-GPC3 therapeutic agent and, (c) extracting circulating tumor DNA (ctDNA) from the same MRCLS patient to which the anti-GPC3 therapeutic agent is administered. Examples of preferred methods for assaying CTCs or ctDNA and has been disclosed in WO 20150/58079; WO 2016/179530; WO 2020/112566; (Cree et al., 2017; Ge et al., 2021; Ono et al., 2015; Yi et al., 2021).

Any of the detecting agents disclosed herein (e.g., anti-GPC3 antibodies) may also be used in the diagnostic assays performed on fluid samples.

II. Treatment of MRCLS with Anti-GPC3 Therapeutic Agents

MRCLS carries an intermediate risk with approximately one-third of patients developing metastases and eventually dying of their tumors. Another feature distinguishing MRCLS from the other types of liposarcoma and most other soft tissue sarcomas is its tendency to metastasize to other soft tissue sites, including the trunk and extremities, the retroperitoneum, the chest wall, the pleura, and the pericardium. Histologically, the MRCLS resection specimens are classified as of purely myxoid type or of myxoid type with a round cell component. Tumors within the purely myxoid subgroup shows a wide morphologic spectrum in terms of cellularity and lipogenic differentiation. Round cell components are either seen as sharply demarcated nodules or as a gradual transition from cellular areas of myxoid liposarcomas. Round cell components are defined as highly cellular areas with prominent primitive round cells with increased nucleocytoplasmic ratio and usually prominent nucleoli. According to the Trojani grading system, purely myxoid liposarcomas are grade 2, while tumors with a prominent round cell component are grade 3 (Haniball et al., 2011). MRCLS is known to be associated with an unusual pattern of metastasis to the bone such as spine and other soft tissues such as retroperitoneum, limb, and axilla whereas other soft tissue sarcomas tend to metastasize to the lung, while other sites are typically involved in advanced stages of the disease. Further, extrapulmonary metastasis was observed in only a minority of MRCLS patients (Asano et al., 2012).

In some aspects, the present disclosure provides a method for treating MRCLS patients with an anti-GPC3 therapeutic agent such as those provided herein. In some embodiments, the method for treating MRCLS patients with an anti-GPC3 therapeutic agent may be staged as grade 1. In another embodiment, the method for treating MRCLS patients with an anti-GPC3 therapeutic agent may be staged as grade 2. In another embodiment, the method for treating MRCLS patients with an anti-GPC3 therapeutic agent may be staged as grade 3. Further, in a preferred embodiment, the method for treating MRCLS patients with an anti-GPC3 therapeutic agent may be staged as advanced and unresectable. In some examples, the anti-GPC3 therapeutic agent are the anti-GPC3 CAR-T cells disclosed herein. In some embodiments, the patients subject to the anti-GPC3 therapy as disclosed herein may be identified using any of the diagnostic methods also disclosed herein.

In some embodiments, a physician may be considering whether to treat the patient with a pharmaceutical product that is indicated for patients whose MRCLS tumor tests positive for GPC3 expression. In one embodiment, the therapeutic agent comprises an anti-GPC3 binding domain, preferably an anti-GPC3 antibody or functional fragment thereof retaining binding to GPC3, preferably wherein the therapeutic agent comprises the administration of an anti-GPC3 antibody, an anti-GPC3 antibody-drug conjugate, an anti-GPC3 antibody-radionuclide conjugate, or a fusion protein of an anti-GPC3 antibody or antibody derivative binding to GPC3 with an anti-CD3 binding domain or an immunostimulatory polypeptide.

In deciding how to use the GPC3 test results in treating any individual patient, the physician may also take into account other relevant circumstances, such as the stage of MRCLS to be treated, the age, weight, gender, genetic background and race of the patient, including inputting a combination of these factors and the test results into a model that helps guide the physician in choosing a therapy and/or treatment regimen with that therapy. Some non-limiting examples of anti-GPC3 therapeutic agents includes chimeric antigen receptor polypeptides, antibody-drug conjugates, bispecific and multispecific. The anti-GPC3 therapeutic agents may be administered intravenously, intradermally, intraperitonially, and/or in encapsulation preferably as an oral composition.

### A. Anti-GPC3 Therapeutic Agents

Any therapeutic agents targeting GPC3 may be used in the methods disclosed herein. In some embodiments, the anti-GPC3 therapeutic agents for use in treating MRCLS as disclosed herein may be an anti-GPC3 antibody. Alternatively, the anti-GPC3 therapeutic agents may be anti-GPC3 chimeric antigen receptor (CAR) and hematopoietic cells such as immune cells (e.g., T cells) expressing such.

### (i) Anti-GPC3 Antibodies

In a non-limiting aspect, examples of an anti-GPC3 antibody that may be used as an anti-GPC3 therapeutic agent of the present invention can include an antibody-drug conjugate (ADC) (WO 2007/137170) comprising a 1G12 antibody (WO 2003/100429) (sold under Cat. No. B0134R by BioMosaics Inc.) conjugated with a cytotoxic toxin. Other examples of anti-GPC3 ADC are disclosed in WO 2017/196764 and CN110577600. Antigen-binding molecules are conjugated with these compounds via appropriate linkers or the like.

In an alternate non-limiting aspect, examples of the anti-GPC3 antibody include a humanized anti-GPC3 antibody described in WO 2006/006693; WO 2009/041062; WO 2013/070468.

In a further alternative non-limiting aspect, examples of the anti-GPC3 antibody include a bispecific comprising anti-GPC3 antibody such as targeting GPC3 & ASGPR1 (WO 2016/086813) and GPC3 & CD40 (WO 2020/230901).

### (ii) Anti-GPC3 CAR and Genetically Modified Hematopoietic Cells Expressing Such

In other embodiments, the anti-GPC3 therapeutic agent disclosed herein may be an anti-GPC3 chimeric antigen receptor (CAR), and hematopoietic cells such as immune cells (e.g., T cells) expressing such an anti-GPC3 CAR, for example, those described in WO 2015/172341; CN105949324 and WO 2016/049459.

CAR polypeptides described herein are used in cell-based immune therapy. The CAR polypeptides described herein may comprise an extracellular domain comprising a scFv with binding affinity to GPC3 and a transmembrane domain, and a CD3(cytoplasmic signaling domain. In some embodiments, a CAR polypeptide as described herein may comprise, from N-terminus to C-terminus, the extracellular antigen binding domain, the transmembrane domain, the optional one or more co-stimulatory domains (e.g., a CD28 co-stimulatory domain, a 4-1BB co-stimulatory signaling domain, an OX40 co-stimulatory signaling domain, a CD27 co-stimulatory signaling domain, or an ICOS co-stimulatory signaling domain; SEQ ID NO: 5, SEQ ID NO: 6, SEQ ID NO: 20, SEQ ID NO: 21, SEQ ID NO: 22), and the CD3(cytoplasmic signaling domain.

Alternatively or in addition, the CAR polypeptides described herein may contain two or more co-stimulatory signaling domains, which may link to each other or be separated by the cytoplasmic signaling domain. The extracellular antigen binding domain, transmembrane domain, optional co-stimulatory signaling domain(s), and cytoplasmic signaling domain in a CAR polypeptide may be linked to each other directly, or via a peptide linker. In some embodiments, any of the CAR polypeptides described herein may comprise a signal sequence at the N-terminus.

In some examples, the modified hematopoietic cells may express a chimeric receptor polypeptide that binds GPC3. Such an anti-GPC3 CAR may comprise (a) an extracellular target binding domain that binds GPC3; (b) a transmembrane domain; and (c) a cytoplasmic signaling domain (e.g., a cytoplasmic domain that comprises an immunoreceptor tyrosine-based activation motif (ITAM)). In some examples, (c) is located at the C-terminus of the chimeric receptor polypeptide. In some instances, the chimeric polypeptide may further comprise at least one co-stimulatory signaling domain. In other instances, the chimeric receptor polypeptide may be free of co-stimulatory signaling domains. In other instances, the CAR polypeptide may be free of co-stimulatory signaling domains. Any of the CAR polypeptides described herein may further comprise a hinge domain, which is located at the C-terminus of (a) and the N-terminus of (b). In other examples, the chimeric receptor polypeptide may be free of any hinge domain. In one embodiment, the extracellular antigen binding domain is a single chain antibody fragment (scFv) that binds to a GPC3, preferably wherein the scFv is derived from the GC33 antibody. In some examples, the scFv has the sequence of SEQ ID NO: 2. In some embodiments, the transmembrane domain of (b) in the CAR can be of a single-pass membrane protein, e.g., CD8α, CD8β, 4-1BB, CD28, CD34, CD4, FcεRIγ, CD16A, OX40, CD3ζ, CD3ε, CD3γ, CD36, TCRα, CD32, CD64, VEGFR2, FAS, and FGFR2B (SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45 respectively). CD16A encompasses the CD16A polymorphism variants CD16^{158F} (SEQ ID NO: 16 and SEQ ID NO: 17) and CD16^{158V} (SEQ ID NO: 18 and SEQ ID NO: 18) (Arriga et al., 2020). Alternatively, the transmembrane domain of (b) can be a non-naturally occurring hydrophobic protein segment. In some embodiments, the at least one co-stimulatory signaling domain of the CAR polypeptides described herein, if applicable, can be of a co-stimulatory molecule, which can be for e.g. 4-1BB, CD28, CD28_{LL→GG} variant, OX40, ICOS, CD27, GITR, HVEM, TIM1, LFA1, and CD2 (SEQ ID NO: 5, SEQ ID NO: 6, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27 respectively).

In some examples, the at least one co-stimulatory signaling domains is a CD28 co-stimulatory signaling domain or a 4-1BB co-stimulatory signaling domain. In some instances, one of the co-stimulatory signaling domains is a CD28 co-stimulatory signaling domain; and the other co-stimulatory domain can be a 4-1BB co-stimulatory signaling domain, an OX40 co-stimulatory signaling domain, a CD27 co-stimulatory signaling domain, or an ICOS co-stimulatory signaling domain. Specific examples include, but are not limited to, CD28 and 4-1BB; or CD28_{LL→GG} variant and 4-1BB. Alternatively, any of the chimeric receptor polypeptide may be free of any co-stimulatory signaling domain. In some embodiments, the CAR polypeptides may further include (i) a CD28 co-stimulatory domain (SEQ ID NO: 6), in combination with a CD28 transmembrane domain, a CD28 hinge domain, or a combination thereof (SEQ ID NO: 4), or (ii) a 4-1BB co-stimulatory domain, preferably SEQ ID NO: 5, in combination with a CD8 transmembrane domain, a CD8 hinge domain, or a combination thereof (SEQ ID NO: 3); more preferably wherein the CAR polypeptide comprises the amino acid sequence of SEQ ID NO: 8 or SEQ ID NO: 9.

In one embodiment, one or more co-stimulatory signaling domains, one of which may be a CD28 co-stimulatory signaling domain or a 4-1BB co-stimulatory signaling domain. The CAR polypeptides are configured such that, when expressed on a host cell, the extracellular antigen-binding domain is located extracellularly for binding to a target molecule and the CD3ζ cytoplasmic signaling domain is located intracellularly for signaling into the cell. The co-stimulatory signaling domain may be located in the cytoplasm for triggering activation and/or effector signaling. In some embodiments, the cytoplasmic signaling domain of (c) in any of the CAR polypeptides described herein can be a cytoplasmic domain of CD3ζ (SEQ ID NO: 7) or FcεR1γ.

In some embodiments, the hinge domain of the CAR polypeptides described herein, when applicable, can be of CD28, CD16A, CD8α, or IgG. In other examples, the hinge domain is a non-naturally occurring peptide. For example, the non-naturally occurring peptide may be an extended recombinant polypeptide (XTEN) or a (Gly₄Ser)ₙ polypeptide, in which n is an integer of 3-12, inclusive. In some examples, the hinge domain is a short segment, which may contain up to 60 amino acid residues.

In specific examples, a CAR polypeptide described herein may comprise (i) a CD28 co-stimulatory domain or a 4-1BB co-stimulatory domain; and (ii) a CD28 transmembrane domain, a CD28 hinge domain, or a combination thereof. In further specific examples, a CAR polypeptide described herein may comprise (i) a CD28 co-stimulatory domain or a 4-1BB co-stimulatory domain, (ii) a CD8 transmembrane domain, a CD8 hinge domain, or a combination thereof.

TABLE 1
Exemplary components of CAR polypeptides.

| | Extracellular | | | Co- | Cytoplasmic |
|---|---|---|---|---|---|
| Signal | domain (antigen | Hinge | Transmembrane | stimulatory | signaling |
| Sequence | binding) | domain | domain | domain | domain |
| CD8α | scFv (e.g., anti- | CD8 | CD8 | 4-1BB | CD3ζ |
| SEQ ID NO: 1 | GPC3 scFv) | SEQ ID NO: 3 | | SEQ ID NO: 5 | SEQ ID NO: 7 |
| | SEQ ID NO: 2 | | | | |
| CD8α | scFv (e.g., anti- | CD28 | CD28 | CD28 | CD3ζ |
| SEQ ID NO: 1 | GPC3 scFv) | SEQ ID NO: 4 | | SEQ ID NO: 6 | SEQ ID NO: 7 |
| | SEQ ID NO: 2 | | | | |

For example, the CAR polypeptide may comprise an amino acid sequence selected from SEQ ID NO: 8 and SEQ ID NO: 9. Amino acid sequences of the example CAR polypeptides are provided in SEQ ID NO: 8, SEQ ID NO: 9, SEQ ID NO: 10 and SEQ ID NO: 11.

In some embodiments, the hematopoietic cells expressing an anti-GPC3 CAR may further express or overly express a factor (e.g., an exogenous factor) that affects glucose metabolism in hematopoietic cells such as immune cells (see WO 2020/037066; WO 2020/097346; and WO 2020/010110, the relevant disclosures of each of which are incorporated by reference herein for the subject matter and purpose referenced herein). Such a factor may be used to divert or re-direct glucose metabolites out of the glycolysis pathway in hematopoietic cells such as immune cells.

In some embodiments, re-direction of glucose metabolites out of the glycolysis pathway may be achieved by expressing (e.g., over-expressing) in hematopoietic cells (e.g., T cells or natural killer cells) one or more factors (e.g., proteins or nucleic acids) such as those described herein. Such genetically engineered hematopoietic cells are expected to have an enhanced metabolic activity relative to native hematopoietic cells of the same type, for example, in a low glucose, low amino acid, low pH, and/or hypoxic environment (e.g., in a tumor microenvironment). As such, hematopoietic cells such as HSCs or immune cells that co-express one or more factors (e.g., polypeptides or nucleic acids) that redirect glucose metabolites out of the glycolysis pathway in the hematopoietic cells and a chimeric receptor polypeptide would exhibit superior bioactivities (e.g., under low glucose, low amino acid, low pH, and/or hypoxic conditions) in the presence of a CAR, for example, cell proliferation, activation (e.g., increased cytokine production, e.g., IL-2 or IFNγ production), cytotoxicity, and/or in vivo anti-tumor activity.

Accordingly, provided herein are modified (e.g., genetically modified) hematopoietic cells (e.g., hematopoietic stem cells, e.g., immune cells such as T cells or natural killer cells) that have a modulated Krebs cycle relative to a native hematopoietic cell of the same type, particularly, for example, in low glucose, low amino acid, low pH, and/or hypoxic conditions. The modified hematopoietic cells may express or overly express a Krebs cycle modulating polypeptide. In some embodiments, the Krebs cycle modulating polypeptide may be an enzyme that catalyzes a reaction of the Krebs cycle. Examples include, but are not limited to, isocitrate dehydrogenase (IDH) such as IDH1 or IDH2, malate dehydrogenase (MDH) such as MDH1 or MDH2, or phosphoglycerate dehydrogenase (PHGDH). In other embodiments, the Krebs cycle modulating polypeptide is an enzyme that uses a Krebs cycle metabolite as a substrate. Examples include, but are not limited to, a glutamic-oxaloacetic transaminase (GOT) such as GOT1 (e.g. SEQ ID NO: 13) or GOT2 (e.g. SEQ ID NO: 12) (also known as aspartate transaminase or aspartate aminotransferase) or phosphoenolpyruvate carboxykinase 1 (PCK1). In yet other embodiments, the Krebs cycle modulating polypeptide is an enzyme that converts a precursor to a Krebs cycle metabolite. Examples include, but are not limited to, a phosphoserine aminotransferase (PSAT1), a glutamate dehydrogenase (GDH1), a glutamic-pyruvate transaminase 1 (GPT1), or a glutaminase (GLS). In specific examples, the polypeptide that redirect glucose metabolites out of the glycolysis pathway used in any of the modified hematopoietic cells such as immune cells can be GOT2 as previously disclosed within WO 2020/037066. Other embodiments may be modified hematopoietic such as immune cells with glucose importation polypeptides such as Glucose transporter (GLUT1, GLUT3) disclosed within WO 2020/010110, or lactate modulators such as monocarboxylate transporter (MCT1, MCT2, MCT4) as disclosed within WO 2020/051493.

In some examples, the hematopoietic cells co-expressing an anti-GPC3 CAR and a factor that affects glucose metabolism may have an improved glucose uptake activity as relative to a wild-type hematopoietic cell of the same type. In some instances, the hematopoietic cell may exogenously express a glucose importation polypeptide, for example, a glucose transporter (GLUT) or a sodium-glucose cotransporter (SGLT). Examples include, but are not limited to, GLUT1, GLUT3, GLUT1 S226D, SGLT1, SGLT2, GLUT8, GLUT8 L12A L13A, GLUT11, GLUT7, and GLUT4.

In other examples, the hematopoietic cells co-expressing an anti-GPC3 CAR and a factor that affects glucose metabolism may have a modulated Krebs cycle as relative to a wild-type hematopoietic cell of the same type. In some instances, the hematopoietic cell may exogenously express a Krebs cycle modulating polypeptide. In some examples, the Krebs cycle modulating factor can be an enzyme that catalyzes a reaction in the Krebs cycle. Examples include, but are not limited to, isocitrate dehydrogenase (IDH), malate dehydrogenase (MDH), or phosphoglycerate dehydrogenase (PHGDH). In other instances, the Krebs cycle modulating polypeptide may be an enzyme that uses a Krebs cycle metabolite as a substrate. Examples include, but are not limited to, glutamic-oxaloacetic transaminase (GOT) or phosphoenolpyruvate carboxykinase 1 (PCK1). In yet other instances, the Krebs cycle modulating polypeptide may be an enzyme that converts a precursor to a Krebs cycle metabolite. Examples include, but are not limited to, phosphoserine aminotransferase (PSATI), glutamate dehydrogenase (GDH1), glutamate-pyruvate transaminase 1 (GPT1), or glutaminase (GLS).

In yet other examples, the hematopoietic cells co-expressing an anti-GPC3 CAR and a factor that affects glucose metabolism may have enhanced intracellular lactate concentrations relative to a wild-type hematopoietic cell of the same type. In some instances, the hematopoietic cell may exogenously express a lactate-modulating polypeptide. In some examples, the lactate modulation polypeptide can be a monocarboxylate transporter (MCT), preferably MCT1, MCT2, or MCT4. In some examples, the lactate-modulating polypeptide may be an enzyme involved in lactate synthesis, for example, lactate dehydrogenase A (LDHA). In yet other examples, the lactate-modulating polypeptide may be a polypeptide that inhibits a pathway that competes for lactate-synthesis substrates, for example, pyruvate dehydrogenase kinase 1 (PDK1).

The Krebs cycle modulating polypeptide may be a naturally-occurring polypeptide from a suitable species, for example, a mammalian Krebs cycle modulating polypeptide such as those derived from human or a non-human primate. Such naturally-occurring polypeptides are known in the art and can be obtained, for example, using any of the above-noted amino acid sequences as a query to search a publicly available gene database, for example GenBank. The Krebs cycle modulating polypeptide for use in the instant disclosure may share a sequence identity of at least 85% (e.g., 90%, 95%, 97%, 98%, 99%, or above) with any of the exemplary proteins GOT1(SEQ ID NO: 13) and GOT2 (SEQ ID NO: 12), preferably with GOT2 (SEQ ID NO: 12).

To construct the hematopoietic cells that express an anti-GPC3 CAR and optionally any of the glucose importation polypeptides described herein, expression vectors for stable or transient expression of the glucose importation polypeptides and/or the chimeric receptor polypeptide may be created via conventional methods as described herein and introduced into immune host cells. For example, nucleic acids encoding the glucose importation polypeptides and/or the chimeric receptor polypeptides may be cloned into one or two suitable expression vectors, such as a viral vector in operable linkage to a suitable promoter. In some instances, each of the coding sequences for the chimeric receptor polypeptide and the glucose importation polypeptide are on two separate nucleic acid molecules and can be cloned into two separate vectors, which may be introduced into suitable host cells simultaneously or sequentially.

Alternatively, the coding sequences for the chimeric receptor polypeptide and the glucose importation polypeptide are on one nucleic acid molecule and can be cloned into one vector. The coding sequences of the chimeric receptor polypeptide and the glucose importation polypeptide may be in operable linkage to two distinct promoters such that the expression of the two polypeptides is controlled by different promoters. Alternatively, the coding sequences of the chimeric receptor polypeptide and the glucose importation polypeptide may be in operable linkage to one promoter such that the expression of the two polypeptides is controlled by a single promoter. Suitable sequences may be inserted between the coding sequences of the two polypeptides so that two separate polypeptides can be translated from a single mRNA molecule. Such sequences, for example, IRES or ribosomal skipping site, are well known in the art.

The nucleic acids and the vector(s) may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the glucose importation polypeptides and/or the chimeric receptor polypeptides. The synthetic linkers may contain nucleic acid sequences that correspond to a particular restriction site in the vector. The selection of expression vectors/plasmids/viral vectors would depend on the type of host cells for expression of the glucose importation polypeptides and/or the chimeric receptor polypeptides, but should be suitable for integration and replication in eukaryotic cells.

A variety of promoters can be used for expression of the glucose importation polypeptides and/or the chimeric receptor polypeptides described herein, including, without limitation, cytomegalovirus (CMV) intermediate early promoter, a viral LTR such as the Rous sarcoma virus LTR, HIV-LTR, HTLV-1 LTR, the simian virus 40 (SV40) early promoter, the human EF1-alpha promoter, or herpes simplex tk virus promoter. Additional promoters for expression of the glucose importation polypeptides and/or the chimeric receptor polypeptides include any constitutively active promoter in a hematopoietic cell. Alternatively, any regulatable promoter may be used, such that its expression can be modulated within a hematopoietic cell.

Additionally, the vector may contain, for example, some or all of the following: a selectable marker gene, such as the neomycin gene or the kanamycin gene for selection of stable or transient transfectants in host cells; enhancer/promoter sequences from the immediate early gene of human CMV for high levels of transcription; intron sequences of the human EF1-alpha gene; transcription termination and RNA processing signals from SV40 for mRNA stability; SV40 polyomavirus origins of replication and Co1E1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase or an inducible caspase such as iCasp9), and reporter gene for assessing expression of the glucose importation polypeptides and/or the chimeric receptor polypeptide.

Suitable vectors and methods for producing vectors containing transgenes are well known and available in the art. Examples of the preparation of vectors for expression of glucose importation polypeptides and/or chimeric receptor polypeptides can be found, for example, in US2014/0106449, herein incorporated in its entirety by reference. Any of the vectors comprising a nucleic acid sequence that encodes a glucose importation polypeptide and/or a chimeric receptor polypeptide described herein is also within the scope of the present disclosure. Such a vector, or the sequence encoding a glucose importation polypeptide and/or a chimeric receptor polypeptide contained therein, may be delivered into host cells such as host hematopoietic cells by any suitable method. Methods of delivering vectors to hematopoietic cells are well known in the art and may include DNA electroporation, RNA electroporation, transfection using reagents such as liposomes, or viral transduction (e.g., retroviral transduction such as lentiviral transduction).

In some embodiments, the vectors for expression of the glucose importation polypeptides and/or the chimeric receptor polypeptides are delivered to host cells by viral transduction (e.g., retroviral transduction such as lentiviral transduction). Exemplary viral methods for delivery include, but are not limited to, recombinant retroviruses (see, e.g., WO 90/07936; WO 94/03622; WO 93/25698; WO 93/25234; WO 93/11230; WO 93/10218; and WO 91/02805; U.S. Pat. Nos. 5,219,740 and 4,777,127; GB2,200,651; and EP0345242, alphavirus-based vectors, and adeno-associated virus (AAV) vectors (see, e.g., WO 94/12649, WO 93/03769; WO 93/19191; WO 94/28938; WO 95/11984; and WO 95/00655). In some embodiments, the vectors for expression of the glucose importation polypeptides and/or the chimeric receptor polypeptides are retroviruses. In some embodiments, the vectors for expression of the glucose importation polypeptides and/or the chimeric receptor polypeptides are lentiviruses. Examples of references describing retroviral transduction include U.S. Pat. No. 5,399,346; (Mann et al., 1983); U.S. Pat. Nos. 4,650,764; 4,980,289; (Markowitz et al., 1988); U.S. Pat. No. 5,124,263; WO 95/07358; (Kuo et al., 1993). WO 95/07358 describes high efficiency transduction of primary B lymphocytes. See also WO 2016/040441 A1, which is incorporated by reference herein for the purpose and subject matter referenced herein.

In examples in which the vectors encoding glucose importation polypeptides and/or chimeric receptor polypeptides are introduced to the host cells using a viral vector, viral particles that are capable of infecting the hematopoietic cells and carry the vector may be produced by any method known in the art and can be found, for example in WO 1991/002805 A2, WO 1998/009271 A1, and U.S. Pat. No. 6,194,191. The viral particles are harvested from the cell culture supernatant and may be isolated and/or purified prior to contacting the viral particles with the hematopoietic cells. In other instances, the nucleic acid encoding the glucose importation polypeptide and the nucleic acid encoding the chimeric receptor polypeptide may be cloned into the same expression vector. Polynucleotides (including vectors in which such polynucleotides are operably linked to at least one regulatory element) for expression of the chimeric receptor polypeptide and glucose importation polypeptide are also within the scope of the present disclosure. Non-limiting examples of useful vectors of the disclosure include viral vectors such as, e.g., retroviral vectors including gamma retroviral vectors, adeno-associated virus vectors (AAV vectors), and lentiviral vectors. In some instances, the nucleic acid(s) encoding the glucose importation polypeptide and/or the chimeric receptor polypeptide may be delivered into host cells via transposons. In some instances, the encoding nucleic acid(s) may be delivered into host cells via gene editing, for example, by CRISPR, TALEN, ZFN, or meganucleases.

In some instances, the nucleic acid described herein may comprise two coding sequences, one encoding a chimeric receptor polypeptide as described herein, and the other encoding a polypeptide capable of enhancing glucose importation (i.e., a glucose importation polypeptide polypeptide). The nucleic acid comprising the two coding sequences described herein may be configured such that the polypeptides encoded by the two coding sequences can be expressed as independent (and physically separate) polypeptides. To achieve this goal, the nucleic acid described herein may contain a third nucleotide sequence located between the first and second coding sequences. This third nucleotide sequence may, for example, encode a ribosomal skipping site. A ribosomal skipping site is a sequence that impairs normal peptide bond formation. This mechanism results in the translation of additional open reading frames from one messenger RNA. This third nucleotide sequence may, for example, encode a P2A, T2A, or F2A peptide (see, for example, (Kim et al., 2011). As a non-limiting example, an exemplary P2A peptide may have the amino acid sequence of ATNFSLLKQAGDVEENPGP (SEQ ID NO: 14). In another embodiment, the third nucleotide sequence may encode an internal ribosome entry site (IRES). An IRES is an RNA element that allows translation initiation in an end-independent manner, also permitting the translation of additional open reading frames from one messenger RNA. Alternatively, the third nucleotide sequence may encode a second promoter controlling the expression of the second polypeptide. The third nucleotide sequence may also encode more than one ribosomal skipping sequence, IRES sequence, additional promoter sequence, or a combination thereof. In some examples, the nucleic acid or the nucleic acid set is comprised within a vector or a set of vectors, which can be an expression vector or a set of expression vectors (e.g., viral vectors such as lentiviral vectors or retroviral vectors). A nucleic acid set, or a vector set refers to a group of two or more nucleic acid molecules or two or more vectors, each encoding one of the polypeptides of interest (i.e., a polypeptide or nucleic acid that redirect glucose metabolites out of the glycolysis pathway and the CAR polypeptide). Any of the nucleic acids described herein is also within the scope of the present disclosure.

Hematopoietic cells described herein, may be immune cells expressing the glucose importation polypeptide may be natural killer cells, monocytes/macrophages, neutrophils, eosinophils, or T cells. Further, the hematopoietic cells, preferably the immune cells, can be obtained from any source, such as peripheral blood mononuclear cells (PBMCs), bone marrow, or tissues such as spleen, lymph node, thymus, stem cells, or tumor tissue. Alternatively, the hematopoietic cells may be derived from stem cells, for example, hematopoietic stem cells and induced pluripotent stem cells (iPSCs). A source suitable for obtaining the type of host cells desired would be evident to one of skill in the art. In some embodiments, the hematopoietic cells, preferably the immune cells, are derived from PBMCs, which may be obtained from a patient (e.g., a human patient) who needs the treatment described herein. As a non-limiting example, anti-CD3, anti-CD28 antibodies IL-2 IL-15, phytohemoagglutinin, or an engineered artificial stimulatory cell or particle may be used for expansion of T cells. In a preferred embodiment, in some examples, the immune cell is a T cell, in which the expression of an endogenous T cell receptor, an endogenous major histocompatibility complex, an endogenous beta-2-microglobulin, or a combination thereof has been inhibited or eliminated. The hematopoietic cells described herein, expressing the factor (e.g., polypeptide or nucleic acid) that redirects glucose metabolites and optionally the chimeric receptor polypeptide, may be a hematopoietic stem cell or a progeny thereof. In some embodiments, the hematopoietic cells can be immune cells such as natural killer cell, monocyte/macrophage, neutrophil, eosinophil, or T cell.

In some embodiments, the hematopoietic cells are natural killer (NK) cells, macrophages, neutrophils, eosinophils, or T cells, preferably wherein the hematopoietic cells are T cells, in which the expression of an endogenous T cell receptor, an endogenous major histocompatibility complex, an endogenous beta-2-microglobulin, or a combination thereof has been inhibited or eliminated; and/or wherein the hematopoietic cells are derived from peripheral blood mononuclear cells (PBMC), hematopoietic stem cells (HSCs), or inducible pluripotent stem cells (iPSCs), preferably wherein the hematopoietic cells are autologous to the patient.

Any of the genetically modified hematopoietic cells (e.g., HSCs or immune cells) described herein may comprise a nucleic acid or a nucleic acid set, which collectively comprises: (a) a first nucleotide sequence encoding the factor (e.g., polypeptide or nucleic acid) that redirects glucose metabolites; and (b) a second nucleotide sequence encoding the chimeric antigen receptor (CAR) polypeptide. The nucleic acid or the nucleic acid set is a DNA and/or RNA molecule or a set of DNA and/or RNA molecules. In some instances, the hematopoietic cell comprises the nucleic acid, which comprises both the first nucleotide sequence and the second nucleotide sequence. In some embodiments, the coding sequence of the factor (e.g., polypeptide or nucleic acid) that redirects glucose metabolites is upstream of that of the CAR polypeptide. In some embodiments, the coding sequence of the CAR polypeptide is upstream of that of the factor that redirects glucose metabolites. Such a nucleic acid may further comprise a third nucleotide sequence located between the first nucleotide sequence and the second nucleotide sequence, wherein the third nucleotide sequence encodes a ribosomal skipping site (e.g., a P2A peptide), an internal ribosome entry site (IRES), or a second promoter.

In some embodiments, the hematopoietic cells may comprise a nucleic acid or a set of nucleic acids (e.g., a DNA molecule or a set of DNA molecules), which collectively comprises:
(a) a first nucleotide sequence encoding the glucose importation polypeptide, the Krebs cycle modulating polypeptide and/or the lactate-modulating polypeptide; and
(b) a second nucleotide sequence encoding the chimeric antigen receptor polypeptide. In some examples, the hematopoietic cells comprise the nucleic acid, which comprises both the first nucleotide sequence and the second nucleotide sequence. In some examples, the nucleic acid may further comprise (c) a third nucleotide sequence located between the first nucleotide sequence and the second nucleotide sequence. The third nucleotide sequence may encode a ribosomal skipping site or comprise an internal ribosome entry site (IRES), or a second promoter. In one example, the third nucleotide sequence encodes a ribosomal skipping site. In one specific example, the ribosomal skipping site is a P2A peptide.

In some embodiments, the nucleic acid or the nucleic acid set may be comprised within a vector or a set of vectors. In some examples, the vector or vector set can be an expression vector or a set of expression vectors. In other examples, the vector or vector set can comprise one or more viral vectors, for example, a lentiviral vector or retroviral vector.

### B. Pharmaceutical Compositions

Any of the anti-GPC3 therapeutic agents as disclosed herein may be formulated into a pharmaceutical composition for use in the treatment methods disclosed herein that are pharmaceutically acceptable. In some embodiments, the pharmaceutical composition may comprise, in addition to the anti-GPC therapeutic agent, suitable carrier, buffer and/or excipients. Preferably, the pharmaceutical composition comprises any of the hematopoietic cells described herein and a pharmaceutically acceptable carrier and excipients.

The phrase “pharmaceutically acceptable”, as used in connection with compositions of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20^{th} Ed. (2000) Lippincott Williams and Wilkins, Ed. K. E. Hoover.

The pharmaceutical compositions of the disclosure may also contain one or more additional active compounds as necessary for the particular indication being treated, preferably those with complementary activities that do not adversely affect each other. Non-limiting examples of possible additional active compounds include, e.g., IL-2 as well as various agents known in the field and listed in the discussion of combination treatments, below.

### C. Treatment of MRCLS

Moreover, provided herein is a method for inhibiting cells expressing GPC3 (e.g., reducing the number of such cells, blocking cell proliferation, and/or suppressing cell activity) in a subject, who may have or suspected of having MRCLS. The method may comprise administering to a subject in need thereof a population of the hematopoietic cells described herein, which may co-express the factor (e.g., polypeptide or nucleic acid) that redirects glucose metabolites and the CAR polypeptide. The subject (e.g., a human patient such as a human patient suffering from a cancer) may have been treated or is being treated with an anti-cancer therapy (e.g., an anti-cancer agent). In some examples, at least some of the cells expressing the target antigen are located in a low-glucose environment, a low-amino acid (e.g., low glutamine) environment, a low-pH environment, and/or a hypoxic environment, for example a tumor microenvironment.

The methods described herein may comprise introducing into the subject a therapeutically effective amount an antibody and a therapeutically effective amount of the genetically engineered hematopoietic cells such as immune cells (e.g., T cells or NK cells), which co-express a gene that improves viability and/or functionality of the hematopoietic cell in the solid tumor microenvironment of the disclosure and the CAR polypeptide of the disclosure. In some examples, the immune cells are autologous. In other examples, the immune cells are allogeneic. In any of the methods described herein, the hematopoietic cells can be activated, expanded, or both ex vivo. In some instances, the immune cells comprise T cells, which are activated in the presence of one or more of an anti-CD3 antibody, an anti-CD28 antibody, IL-2, IL-15, phytohemoagglutinin, and an engineered artificial stimulatory cell or particle. In other instances, the hematopoietic cells comprise natural killer cells, which are activated in the presence of one or more of 4-1BB ligand, anti-4-1BB antibody, IL-15, anti-IL-15 receptor antibody, IL-2, IL-12, IL-21 and K562 cells, an engineered artificial stimulatory cell or particle.

In some embodiments, the hematopoietic cells are administered to a subject in an amount effective in inhibiting cells expressing the target antigen by least 20% and/or by at least 2-fold, e.g., inhibiting cells expressing the target antigen by 50%, 80%, 100%, 2-fold, 5-fold, 10-fold, 20-fold, 50-fold, 100-fold, or more. The efficacy of the cell-based immunotherapy as described herein may be assessed by any method known in the art and would be evident to a skilled medical professional. For example, the efficacy of the cell-based immunotherapy may be assessed by survival of the subject or tumor or cancer burden in the subject or tissue or sample thereof. In some embodiments, the hematopoietic cells are administered to a subject in need of the treatment in an amount effective in enhancing the efficacy of an cell-based immunotherapy by at least 10% and/or by at least 2-fold, e.g., enhancing the efficacy of an immunotherapy by 50%, 80%, 100%, 2-fold, 5-fold, 10-fold, 20-fold, 50-fold, 100-fold or more, as compared to the efficacy using the same type of hematopoietic cells that do not express the glucose importation polypeptide.

In any of the compositions or methods described herein, the hematopoietic cells (e.g., NK and/or T cells) may be autologous to the subject, i.e., the hematopoietic cells may be obtained from the subject in need of the treatment. Alternatively, the host cells are allogeneic cells, i.e., the cells are obtained from a first subject, genetically engineered as described herein, and administered to a second subject that is different from the first subject but of the same species. Either autologous or allogeneic hematopoietic cells may be activated and/or expanded ex vivo prior to the delivery to the subject.

In accordance with the present disclosure, patients can be treated by infusing therapeutically effective doses of hematopoietic cells such as T lymphocytes or NK cells comprising a glucose importation polypeptide and/or a CAR polypeptide of the disclosure in the range of about 104 to 1010 or more cells per kilogram of body weight (cells/kg). The infusion can be repeated as often and as many times as the patient can tolerate until the patient does not further respond to the treatment, e.g., progressive disease is diagnosed. The appropriate infusion dose and schedule will vary from patient to patient, but can be determined by the treating physician for a particular patient. In a preferred embodiment, at least about 5×10⁴ anti-GPC3−CAR T cells per kg are administered to the selected patient with MRCLS, preferably from about 5×10⁴ to about 1×10¹² anti-GPC3-CAR cells/kg are administered to the patient with MRCLS.

The efficacy of the compositions or methods described herein may be assessed by any method known in the art and would be evident to a skilled medical professional. For example, the efficacy of the compositions or methods described herein may be assessed by survival of the subject or cancer burden in the subject or tissue or sample thereof. In some embodiments, the compositions and methods described herein may be assessed based on the safety or toxicity of the therapy (e.g., administration of the GPC3 targeted hematopoietic cells as described herein, antibody-drug conjugates, bispecific or multi-specific targeting GPC3) in the subject, for example, by the overall health of the subject and/or the presence of adverse events or severe adverse events. In one embodiment, the administration of the anti-GPC3 therapeutic agent is effective in reducing tumor size by at least 10% as measured by computerized tomography (CT) scan. In another embodiment, the administration of the anti-GPC3 therapeutic agent is effective if stable disease according to RECIST (e.g., RECIST 1.1) is achieved, i.e., the sum of total tumor diameters may have an increase by 19% or a decrease by 29%, without new measurable lesions. Preferably, an objective response according to RECIST (e.g., RECIST 1.1) is achieved, i.e., of the total tumor diameters decreased by 30% or more without new measurable lesions. Preferably, tumors are staged by computerized tomography (CT) scan. In another aspect, resectable tumors are staged histologically.

In some examples, the subject to be treated by the methods described herein is a human patient suffering from MRCLS staged as grade 1, grade 2, grade 3, for example, metastatic MRCLS or advanced unresectable MRCLS. In one embodiment, GPC3 specific CAR-Ts are administered into patient suffering from MRCLS staged as grade 1. In another embodiment, GPC3 specific CAR-Ts are administered into patient suffering from MRCLS staged as grade 2. Further, in another embodiment, GPC3 specific CAR-Ts are administered into patient suffering from MRCLS staged as grade 3. In a preferred embodiment, GPC3 specific CAR-Ts are administered into patient suffering from MRCLS, for example, metastatic MRCLS or advanced unresectable MRCLS.

Also, within the scope of the present disclosure are uses of the anti-GPC3 therapeutic agent described herein, for treating MRCLS, and uses thereof for manufacturing a medicament for the intended medical treatment.

In some embodiments, the genetically engineered hematopoietic cells, expressing a gene that improves viability and/or functionality of the hematopoietic cell in the solid tumor microenvironment of the disclosure, may be derived from natural hematopoietic cells specific to MRCLS cells (e.g., MRCLS cells). Such genetically engineered hematopoietic cells (e.g., tumor-infiltrating lymphocytes or TILs) may not co-express any chimeric receptor polypeptide and can be used to destroy the target disease cells, e.g., MRCLS cells. These genetically engineered TILs, expressing said gene improving viability and/or functionality but not chimeric receptors, may be co-used with a bispecific antibody capable of binding to the target tumor cells and the TILs (BiTE).

Further, the compositions and methods described in the present disclosure may be utilized in conjunction with other types of therapy for cancer, such as chemotherapy, surgery, radiation, gene therapy, and so forth, preferably with the established standard of care for e.g. doxorubicin, ifosfamide, trabectedin as disclosed in (Abaricia & Hirbe, 2018; Lee et al., 2018; Regina & Hettmer, 2019; Sanfilippo et al., 2013; Suarez-Kelly et al., 2019) for MRCLS. Such therapies can be administered simultaneously or sequentially (in any order) with the immunotherapy according to the present disclosure.

When co-administered with an additional therapeutic agent, suitable therapeutically effective dosages for each agent may be lowered due to the additive action or synergy. The treatments of the disclosure can be combined with other immunomodulatory treatments such as, e.g., therapeutic vaccines (including but not limited to GVAX, DC-based vaccines, etc.), checkpoint inhibitors (including but not limited to agents that block CTLA-4, PD-1, LAG-3, TIM-3, etc.) or activators (including but not limited to agents that enhance 41BB, OX40, etc.).

Non-limiting examples of other therapeutic agents useful for combination with the immunotherapy of the disclosure include: (i) anti-angiogenic agents (e.g., TNP-470, platelet factor 4, thrombospondin-1, tissue inhibitors of metalloproteases (TIMP1 and TIMP2), prolactin (16-kD fragment), angiostatin (38-kD fragment of plasminogen), endostatin, bFGF soluble receptor, transforming growth factor beta, interferon alpha, soluble KDR and FLT-1 receptors, placental proliferin-related protein, as well as those listed by (Carmeliet & Jain, 2000); (ii) a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof; and (iii) chemotherapeutic compounds such as, e.g., pyrimidine analogs (5-fluorouracil, floxuridine, capecitabine, gemcitabine and cytarabine), purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones, and navelbine, epidipodophyllotoxins (etoposide and teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamine oxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracylines, mitoxantrone, bleomycin, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antipro-liferative/anti-mitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (brefeldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); AKT inhibitors (such as MK-22062HC1, Perifosine (KRX-0401), GSK690693, Ipatasertib (GDC-0068), AZD5363, uprosertib, afuresertib, or triciribine); mTOR inhibitors, topoisomerase inhibitors (doxorubicin (adriamycin), amsacrine, camptothecin, daunorubicin, dactinomycin, eniposide, epirubicin, etoposide, idarubicin, mitoxantrone, topotecan, and irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors.

In one embodiment, the method further comprises administering at least one immunomodulatory agent to the patient in parallel or sequential to the therapeutic agent, preferably where in the immunomodulatory agent is an immune checkpoint inhibitor or an immunostimulatory cytokine. It is expected that immune checkpoint inhibitors would lift inhibitory signals in the microtumor environment that could negatively interfere with the mode of action of an anti-GPC3 therapeutic agent, e.g., with a hematopoietic cell (e.g., an immune cell such as a T cell or NK cell) expressing a CAR, as such immune checkpoints may tune down the activation of the hematopoietic cells and thereby lower or block their activity. In some embodiments, the method further comprises administering a lymphocyte reduction treatment, preferably selected from cyclophosphamide and fludarabine. Such lymphodepletion treatment is preferably applied prior to the infusion of the hematopoietic cells expressing a CAR in order to allow for greater T cell expansion of the infused cells (Shank et al., 2017).

The details of one or more embodiments of the disclosure are set forth in the description below. Other features or advantages of the present disclosure will be apparent from the detailed description of several embodiments and from the appended claims.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Black-well, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclo-nal antibodies: a practical approach (P. Shepherd and C. Dean, eds., Oxford University Press, 2000); Using antibod-ies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985«; Transcription and Translation (B. D. Hames & S. J. Higgins, eds. (1984«; Animal Cell Culture (R. I. Freshney, ed. (1986«; Immobi-lized Cells and Enzymes (IRL Press, (1986«; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

The following examples are intended only to illustrate methods and embodiments in accordance with the invention, and as such should not be construed as imposing limitations upon the claims.

### Example 1. IHC Assay of FFPE Tissue Sections with Anti-GPC3 Monoclonal Antibody GC33

IHC staining with antibody specific for GPC3 was performed according to conventional protocols. Human biopsy specimens (tumor & healthy tissue), xenograft biopsy specimens (tumor & healthy tissue) and cell line specimen were fixed in neutral buffered 10% formalin solution for 24 hours followed by embedding in paraffin as per standard procedures. The standard sample size was 0.5 cm×1 cm×1 cm. Tissue sections of 5 μm of thickness were cut on a microtome (Leica) and mounted on positively charged slides. Slides were air-dried and stored throughout the duration of the study at RT. Tissue sections were deparaffinized in EZ prep (Ventana), followed by antigen retrieval with Target Retrieval Solution (Ventana) in a heated water bath (98° C., 60 mins). Endogenous peroxidases were blocked with primary peroxidase inhibitor (Ventana) at RT for 5 mins. Thereafter, sections were incubated with the primary anti-GPC3 antibody (antibody GC33; Ventana #790-4564) for 32 mins followed by the revelation of enzymatic activity (OptiView DAB Detection Kit, Ventana). Sections were counterstained with hematoxylin (Ventana) at RT for 30 secs. The specificity of the staining was determined using appropriate isotype controls. Images of whole tumor sections were acquired using a Leica Aperio AT2 scanner (Leica).

### Example 2. IF Assay of FFPE Tissue Sections with Anti-GPC3 Monoclonal Antibody GC33

IF staining with antibody specific for GPC3 is performed according to conventional protocols. Human biopsy specimens (tumor & healthy tissue), xenograft biopsy specimens (tumor & healthy tissue) and cell line specimen are fixed in neutral buffered 10% formalin solution for 24 hours followed by embedding in paraffin as per standard procedures. The standard sample size is 0.5 cm×1 cm×1 cm. Tissue sections of 5 μm of thickness is cut on a microtome (Leica) and mounted on positively charged slides. Slides are air-dried and stored throughout the duration of the study at RT. Briefly, tissue sections are deparaffinized and rehydrated descending alcohol series (100, 96, 70, and 50%), followed by antigen retrieval with Target Retrieval Solution (Leica) in preheated water bath (97° C., 30 mins). Sections are cooled down to RT for 30 mins. Sections are then treated with Signal enhancer (Fisher Thermoscientific) at RT for 30 min followed by blocking buffer at RT for 60 mins. The anti-GPC3 antibody (antibody GC33; Ventana #790-4564) is applied at RT for 2 hours. Next, the slides are incubated with appropriate fluorophore-labelled secondary antibodies at RT for 1 hour. Finally, sections are treated with TrueBlack® Lipofuscin Autofluorescence Quencher (Biotium) for 30 secs and mounted with ProLong Gold antifade reagent containing DAPI (Thermo Fisher Scientific). The specificity of the staining is determined using appropriate isotype controls. Images of whole tumor sections were acquired using a Leica Aperio AT2 scanner (Leica).

### Example 3. IHC Scoring of FFPE Tissue Sections from Different Cancer Types for GPC3 Expression

Example 1 was scored for GPC3 expression using a detailed scoring process. The brief description of the figures lists the various scores that were assigned to individual fields of each tissue section for illustrative purposes only. However, a scoring process was performed by examining the entire tissue section on the slide, and in practice, the pathologist scored a slide for GPC3 expression by viewing the tissue section on the slide at low, medium and high magnification. Low and medium power was used to detect stained tumor cells. Medium and high power was used to examine individual tumor cells to estimate the number and intensity of viable tumor cells that exhibit at least partial membrane and cytoplasmic staining. Each stained tissue section was assigned a H-score. The H-score comprised (i) estimating, across all of the viable tumor cells in all of the examined stained tissue section, four separate percentages for cells that have no staining, weak staining (+1), moderate staining (+2) and strong staining (+3), wherein a cell must have at least partial membrane and/or cytoplasmic staining to be included in the weak, moderate or strong staining percentages, and wherein the sum of all four percentages equals 100; and (ii) inputted the estimated percentages into the formula of 1*(percentage of tumor cells with 1+ staining intensity)+2*(percentage of tumor cells with 2+ staining intensity)+3*(percentage of tumor cells with 1+ staining intensity), and assigned the result of the formula to the tissue section as the H-score.

### Example 4. IF scoring of FFPE tissue sections from different cancer types for GPC3 expression

Example 2 may be scored for GPC3 expression using a detailed scoring process. The brief description of the figures lists the various scores that were assigned to individual fields of each tissue section for illustrative purposes only. However, a scoring process is performed by examining the entire tissue section on the slide, and in practice, the pathologist scores a slide for GPC3 expression by viewing the tissue section on the slide at low, medium and high magnification. Low and medium power is used to detect stained tumor cells. Medium and high power is used to examine individual tumor nests to estimate the number and intensity of viable tumor cells that exhibit at least partial membrane and cytoplasmic staining. Each stained tissue section is assigned a H-score. The H-score comprises (i) estimating, across all of the viable tumor cells in all of the examined stained tissue section, four separate percentages for cells that have no staining, weak staining (+1), moderate staining (+2) and strong staining (+3), wherein a cell must have at least partial membrane and/or cytoplasmic staining to be included in the weak, moderate or strong staining percentages, and wherein the sum of all four percentages equals 100; and (ii) inputting the estimated percentages into the formula of 1*(percentage of tumor cells with 1+ staining intensity)+2*(percentage of tumor cells with 2+ staining intensity)+3*(percentage of tumor cells with 1+ staining intensity), and assigning the result of the formula to the tissue section as the H-score.

### Example 5. ISH Assay of FFPE Tissue Sections with Probes Specific for GPC3

In situ hybridization staining for target gene GPC3 may be performed according to conventional protocols (Wang et al., 2012). Human biopsy specimens (tumor & healthy tissue), xenograft biopsy specimens (tumor & healthy tissue) and cell line specimen are fixed in neutral buffered 10% formalin solution for 24 hours followed by embedding in paraffin as per standard procedures. The standard sample size is 0.5 cm×1 cm×1 cm. Tissue sections of 6 μm of thickness is cut on a microtome (Leica) and mounted on positively charged slides. Tissue quality for each sample is assessed by performing RNA hybridization for mRNA of the housekeeping gene Ubiquitin C (UBC) according to conventional protocols (Wang et al., 2012). The protocol begins with slides that are air-dried and stored throughout the duration of the study at RT. Briefly, tissue sections are deparaffinized and rehydrated descending alcohol series (100, 96, 70, and 50%), followed by air-drying at RT for 5 mins. The slides are pre-treated in pre-hybridization buffer at 40° C. for 30 mins followed by quenching peroxidase at RT for 10 mins (ACD Biotechnie). The slides are submerged in target retrieval at 40° C. for 30 mins followed by protease treatment at 40° C. for 30 mins. The slides are incubated with target probe and incubated at 40° C. for 2 hrs followed by washing off excess probes in appropriate buffers (ACD Biotechnie). Detection of the probe is carried, e.g., in fast red solution in case of chromogenic assay by incubating at RT for 10 mins. Sections are counterstained with hematoxylin (Ventana) at RT for 30 secs. The specificity of the staining is determined using appropriate isotype controls. Images of whole tumor sections are acquired using a Leica Aperio AT2 scanner (Leica).

### Example 6. Isolation of Cell-Free DNA (cfDNA) and Analysis by Targeted Next Generation Sequencing (NGS)

Blood samples may be isolated pre- or post-surgery of suspected patients. Further, they may be matched with patients of primary and/or secondary tumor tissue biopsies. Blood samples may be isolated and processed within 24 h after collection. Blood is first centrifuged at 1700 g for 10 min to separate plasma and blood cells. The separated plasma is centrifuged at 12,000 g for another 10 min to remove cellular debris. The plasma is collected and aliquoted in vials per 2 ml and stored at −80° C. until further processing. cfDNA is isolated from 440 μl to 4 ml (median 3.95 ml) plasma using Circulating Nucleic Acid kit (Qiagen) and followed by elution in 30 μl elution buffer. ctDNA concentrations are determined by Qubit™ 1×dsDNA HS Assay kit (Thermo Fisher scientific) using 2 μl of ctDNA.

Sequencing may be performed by ion semiconductor sequencing on the Ion Torrent S5XL Next generation sequencing (NGS) system using the ctDNA Assay with molecular barcoding loaded on Ion 540 chips. Experiments are performed according to the manufacturer's protocol (Thermo Fisher Scientific/Life Technologies). Several ctDNA panel may be used to cover mutational hotspots of multiple genes relevant to MRCLS (for e.g., Oncomine™ Colon ctDNA panel, Thermo Fisher Scientific/Life Technologies). Analysis and cut-off are performed according to (Ge et al., 2021).

### Example 7. Isolation of Circulating Tumor Cell RNA (ctRNA) and Analysis by RT-PCR

Circulating tumor cells (CTCs) may be isolated from 7.5 ml whole blood on basis of density gradient. Equal volume of whole blood and PBS is mixed carefully by inversion, overlayed on Ficoll-paque followed by centrifugation at 400 g for 30 min at RT. The CTCs are retrieved from the plasma layer (see (Low & Wan Abas, 2015). RNA is extracted from the CTCs as per manufacturer's protocol (QIAGEN). Reverse transcription may be carried out using the Invitrogen Superscript III reverse transcriptase and random hexamers as primers (Invitrogen) and performed at 37° C. for 1 h followed by inactivation at 95° C. for 5 min. cDNA (5 μl) is used in subsequent PCR reactions. GPC3-specific primers and PCR reaction is carried out as described in (Wang et al., 2011).

SEQUENCES
CD8α signal sequence
SEQ ID NO: 1
MALPVTALLLPLALLLHAARP
GPC3 scFv derived from GC33
SEQ ID NO: 2
DVVMTQSPLSLPVTPGEPASISCRSSQSLVHSNRNTYLHWYLQKPGQSPQLLIYKVSNRFSGVPDRFS
GSGSGTDFTLKISRVEAEDVGVYYCSQNTHVPPTFGQGTKLEIKRGGGGSGGGGSGGGGSQVQLVQSG
AEVKKPGASVKVSCKASGYTFTDYEMHWVRQAPGQGLEWMGALDPKTGDTAYSQKFKGRVTLTADKST
STAYMELSSLTSEDTAVYYCTRFYSYTYWGQGTLVTVSS
CD8 hinge and transmembrane domain
SEQ ID NO: 3
TTTPAPRPPTPAPTIASQPLSLRPEACRPAAGGAVHTRGLDFACDIYIWAPLAGTCGVLLLSLVITLY
C
CD28 hinge and transmembrane domain
SEQ ID NO: 4
IEVMYPPPYLDNEKSNGTIIHVKGKHLCPSPLFPGPSKPFWVLVVVGGVLACYSLLVTVAFIIFWVRS
KRSRLLHSDYMNMTPRRPGPTRKHYQPYAPPRDFAAYRS
4-1BB co-stimulatory domain
SEQ ID NO: 5
KRGRKKLLYIFKQPFMRPVQTTQEEDGCSCRFPEEEEGGCEL
CD28 co-stimulatory domain
SEQ ID NO: 6
RSKRSRLLHSDYMNMTPRRPGPTRKHYQPYAPPRDFAAYRS
CD3zeta signaling domain
SEQ ID NO: 7
RVKFSRSADAPAYQQGQNQLYNELNLGRREEYDVLDKRRGRDPEMGGKPRRKNPQEGLYNELQKDKMA
EAYSEIGMKGERRRGKGHDGLYQGLSTATKDTYDALHMQALPPR
GPC3 CAR polypeptide (with signal sequence italicized) (CD8α/GC33 scFv/
CD8α-CD8α/4-1BB/CD3ζ)
SEQ ID NO: 8
MALPVTALLLPLALLLHAARPDVVMTQSPLSLPVTPGEPASISCRSSQSLVHSNRNTYLHWYLQKPGQ
SPQLLIYKVSNRFSGVPDRFSGSGSGTDFTLKISRVEAEDVGVYYCSQNTHVPPTFGQGTKLEIKRGG
GGSGGGGSGGGGSQVQLVQSGAEVKKPGASVKVSCKASGYTFTDYEMHWVRQAPGQGLEWMGALDPKT
GDTAYSQKFKGRVTLTADKSTSTAYMELSSLTSEDTAVYYCTRFYSYTYWGQGTLVTVSSTTTPAPRP
PTPAPTIASQPLSLRPEACRPAAGGAVHTRGLDFACDIYIWAPLAGTCGVLLLSLVITLYCKRGRKKL
LYIFKQPFMRPVQTTQEEDGCSCRFPEEEEGGCELRVKFSRSADAPAYQQGQNQLYNELNLGRREEYD
VLDKRRGRDPEMGGKPRRKNPQEGLYNELQKDKMAEAYSEIGMKGERRRGKGHDGLYQGLSTATKDTY
DALHMQALPPR
mature GPC3 CAR polypeptide (GC33 scFv/CD8α-CD8α/4-1BB/CD3ζ)
SEQ ID NO: 9
DVVMTQSPLSLPVTPGEPASISCRSSQSLVHSNRNTYLHWYLQKPGQSPQLLIYKVSNRFSGVPDRFS
GSGSGTDFTLKISRVEAEDVGVYYCSQNTHVPPTFGQGTKLEIKRGGGGSGGGGSGGGGSQVQLVQSG
AEVKKPGASVKVSCKASGYTFTDYEMHWVRQAPGQGLEWMGALDPKTGDTAYSQKFKGRVTLTADKST
STAYMELSSLTSEDTAVYYCTRFYSYTYWGQGTLVTVSSTTTPAPRPPTPAPTIASQPLSLRPEACRP
AAGGAVHTRGLDFACDIYIWAPLAGTCGVLLLSLVITLYCKRGRKKLLYIFKQPFMRPVQTTQEEDGC
SCRFPEEEEGGCELRVKFSRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDALHMQALPPR
GPC3-CAR polypeptide (with signal sequence italicized) (CD8α/GC33
scFv/CD28-CD28/CD3ζ) - no stimulation domain
SEQ ID NO: 10
MALPVTALLLPLALLLHAARPDVVMTQSPLSLPVTPGEPASISCRSSQSLVHSNRNTYLHWYLQKPGQ
SPQLLIYKVSNRFSGVPDRFSGSGSGTDFTLKISRVEAEDVGVYYCSQNTHVPPTFGQGTKLEIKRGG
GGSGGGGSGGGGSQVQLVQSGAEVKKPGASVKVSCKASGYTFTDYEMHWVRQAPGQGLEWMGALDPKT
GDTAYSQKFKGRVTLTADKSTSTAYMELSSLTSEDTAVYYCTRFYSYTYWGQGTLVTVSSIEVMYPPP
YLDNEKSNGTIIHVKGKHLCPSPLFPGPSKPFWVLVVVGGVLACYSLLVTVAFIIFWVRSKRSRLLHS
DYMNMTPRRPGPTRKHYQPYAPPRDFAAYRSRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTATKDTYDALH
MQALPPR
mature GPC3-CAR polypeptide (GC33 scFv/CD28-CD28/CD3ζ) - no
stimulation domain
SEQ ID NO: 11
DVVMTQSPLSLPVTPGEPASISCRSSQSLVHSNRNTYLHWYLOKPGQSPQLLIYKVSNRFSGVPDRFS
GSGSGTDFTLKISRVEAEDVGVYYCSQNTHVPPTFGQGTKLEIKRGGGGSGGGGSGGGGSQVQLVQSG
AEVKKPGASVKVSCKASGYTFTDYEMHWVRQAPGQGLEWMGALDPKTGDTAYSQKFKGRVTLTADKST
STAYMELSSLTSEDTAVYYCTRFYSYTYWGQGTLVTVSSIEVMYPPPYLDNEKSNGTIIHVKGKHLCP
SPLFPGPSKPFWVLVVVGGVLACYSLLVTVAFIIFWVRSKRSRLLHSDYMNMTPRRPGPTRKHYQPYA
PPRDFAAYRSRVKFSRSADAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR
GOT2
SEQ ID NO: 12
MALLHSGRVLPGIAAAFHPGLAAAASARASSWWTHVEMGPPDPILGVTEAFKRDTNSKKMNLGVGAYR
DDNGKPYVLPSVRKAEAQIAAKNLDKEYLPIGGLAEFCKASAELALGENSEVLKSGRFVTVQTISGTG
ALRIGASFLQRFFKFSRDVFLPKPTWGNHTPIFRDAGMQLQGYRYYDPKTCGFDFTGAVEDISKIPEQ
SVLLLHACAHNPTGVDPRPEQWKEIATVVKKRNLFAFFDMAYQGFASGDGDKDAWAVRHFIEQGINVC
LCQSYAKNMGLYGERVGAFTMVCKDADEAKRVESQLKILIRPMYSNPPLNGARIAAAILNTPDLRKQW
LQEVKVMADRIIGMRTQLVSNLKKEGSTHNWQHITDQIGMFCFTGLKPEQVERLIKEFSIYMTKDGRI
SVAGVTSSNVGYLAHAIHQVTK
GOT1
SEQ ID NO: 13
MAPPSVFAEVPQAQPVLVFKLTADFREDPDPRKVNLGVGAYRTDDCHPWVLPVVKKVEQKIANDNSLN
HEYLPILGLAEFRSCASRLALGDDSPALKEKRVGGVQSLGGTGALRIGADFLARWYNGTNNKNTPVYV
SSPTWENHNAVFSAAGFKDIRSYRYWDAEKRGLDLQGFLNDLENAPEFSIVVLHACAHNPTGIDPTPE
QWKQIASVMKHRFLFPFFDSAYQGFASGNLERDAWAIRYFVSEGFEFFCAQSFSKNFGLYNERVGNLT
VVGKEPESILQVLSQMEKIVRITWSNPPAQGARIVASTLSNPELFEEWTGNVKTMADRILTMRSELRA
RLEALKTPGTWNHITDQIGMFSFTGLNPKQVEYLVNEKHIYLLPSGRINVSGLTTKNLDYVATSIHEA
VTKIQ
P2A
SEQ ID NO: 14
ATNFSLLKQAGDVEENPGP
GLUT1
SEQ ID NO: 15
MEPSSKKLTGRLMLAVGGAVLGSLQFGYNTGVINAPQKVIEEFYNQTWVHRYGESILPTTLTTLWSLS
VAIFSVGGMIGSFSVGLFVNRFGRRNSMLMMNLLAFVSAVLMGFSKLGKSFEMLILGRFIIGVYCGLT
TGFVPMYVGEVSPTALRGALGTLHQLGIWGILIAQVFGLDSIMGNKDLWPLLLSIIFIPALLQCIVLP
FCPESPRFLLINRNEENRAKSVLKKLRGTADVTHDLQEMKEESRQMMREKKVTILELFRSPAYRQPIL
IAWLQLSQQLSGINAVFYYSTSIFEKAGVQQPVYATIGSGIVNTAFTWSLFWERAGRRTLHLIGLAGM
AGCAILMTIALALLEQLPWMSYLSIVAIFGFVAFFEVGPGPIPWFIVAELFSQGPRPAAIAVAGFSNW
TSNFIVGMCFQYVEQLCGPYVFIIFTVLLVLFFIFTYFKVPETKGRTFDEIASGFRQGGASQSDKTPE
ELFHPLGADSQV
CD16A1^{158F} polypeptide (with signal sequence italicized)
SEQ ID NO: 16
MWQLLLPTALLLLVSAGMRTEDLPKAVVFLEPQWYRVLEKDSVTLKCQGAYSPEDNSTQWFHNESLIS
SQASSYFIDAATVDDSGEYRCQTNLSTLSDPVQLEVHIGWLLLQAPRWVFKEEDPIHLRCHSWKNTAL
HKVTYLQNGKGRKYFHHNSDFYIPKATLKDSGSYFCRGLFGSKNVSSETVNITITQGLAVSTISSFFP
PGYQVSFCLVMVLLFAVDTGLYFSVKTNIRSSTRDWKDHKFKWRKDPQDK
mature CD16A^{158F} polypeptide
SEQ ID NO: 17
GMRTEDLPKAVVFLEPQWYRVLEKDSVTLKCQGAYSPEDNSTQWFHNESLISSQASSYFIDAATVDDS
GEYRCQTNLSTLSDPVQLEVHIGWLLLQAPRWVFKEEDPIHLRCHSWKNTALHKVTYLQNGKGRKYFH
HNSDFYIPKATLKDSGSYFCRGLFGSKNVSSETVNITITQGLAVSTISSFFPPGYQVSFCLVMVLLFA
VDTGLYFSVKTNIRSSTRDWKDHKFKWRKDPQDK
CD16A ^{158V} polypeptide (with signal sequence italicized)
SEQ ID NO: 18
MWQLLLPTALLLLVSAGMRTEDLPKAVVFLEPQWYRVLEKDSVTLKCQGAYSPEDNSTQWFHNESLIS
SQASSYFIDAATVDDSGEYRCQTNLSTLSDPVQLEVHIGWLLLQAPRWVFKEEDPIHLRCHSWKNTAL
HKVTYLQNGKGRKYFHHNSDFYIPKATLKDSGSYFCRGLVGSKNVSSETVNITITQGLAVSTISSFFP
PGYQVSFCLVMVLLFAVDTGLYFSVKTNIRSSTRDWKDHKFKWRKDPQDK
mature CD16A^{158V}
SEQ ID NO: 19
GMRTEDLPKAVVFLEPQWYRVLEKDSVTLKCQGAYSPEDNSTQWFHNESLISSQASSYFIDAATVDDS
GEYRCQTNLSTLSDPVQLEVHIGWLLLQAPRWVFKEEDPIHLRCHSWKNTALHKVTYLQNGKGRKYFH
HNSDFYIPKATLKDSGSYFCRGLVGSKNVSSETVNITITQGLAVSTISSFFPPGYQVSFCLVMVLLFA
VDTGLYFSVKTNIRSSTRDWKDHKFKWRKDPQDK
OX-40 co-stimulatory domain
SEQ ID NO: 20
ALYLLRRDQRLPPDAHKPPGGGSFRTPIQEEQADAHSTLAKI
CD27 co-stimulatory domain
SEQ ID NO: 21
QRRKYRSNKGESPVEPAEPCHYSCPREEEGSTIPIQEDYRKPEPACSP
ICOS co-stimulatory domain
SEQ ID NO: 22
KKKYSSSVHDPNGEYMFMRAVNTAKKSRLTDVTL
GITR co-stimulatory domain
SEQ ID NO: 23
QLGLHIWQLRSQCMWPRETQLLLEVPPSTEDARSCQFPEEERGERSAEEKGRLGDLWV
HVEM co-stimulatory domain
SEQ ID NO: 24
CVKRRKPRGDVVKVIVSVQRKRQEAEGEATVIEALQAPPDVTTVAVEETIPSFTGRSPNH
TIM1 co-stimulatory domain
SEQ ID NO: 25
KKYFFKKEVQQLSVSFSSLQIKALQNAVEKEVQAEDNIYIENSLYATD
LFA-1 co-stimulatory domain
SEQ ID NO: 26
DIYIWAPLAGTCGVLLLSLVITLYCYKVGFFKRNLKEKMEAGRGVPNGIPAEDSEQLASGQEAGDPGC
LKPLHEKDSESGGGKD
CD2 co-stimulatory domain
SEQ ID NO: 27
DIYIWAPLAGTCGVLLLSLVITLYCKRKKQRSRRNDEELETRAHRVATEERGRKPHQIPASTPQNPAT
SQHPPPPPGHRSQAPSHRPPPPGHRVQHQPQKRPPAPSGTQVHQQKGPPLPRPRVQPKPPHGAAENSL
SPSSN
CD8α Transmembrane domain
SEQ ID NO: 28
DIYIWAPLAGTCGVLLLSLVITLYC
CD8β Transmembrane domain
SEQ ID NO: 29
DITLGLLVAGVLVLLVSLGVAIHLC
4-IBB Transmembrane domain
SEQ ID NO: 30
DIISFFLALTSTALLFLLFFLTLRFSVV
CD28Transmembrane domain
SEQ ID NO: 31
DFWVLVVVGGVLACYSLLVTVAFIIFWVRS
CD34 Transmembrane domain
SEQ ID NO: 32
DLIALVTSGALLAVLGITGYFLMNR
CD4 Transmembrane domain
SEQ ID NO: 33
DMALIVLGGVAGLLLFIGLGIFFCVR
FcεRIγ Transmembrane domain
SEQ ID NO: 34
DLCYILDAILFLYGIVLTLLYCRLK
OX-40 Transmembrane domain
SEQ ID NO: 35
DVAAILGLGLVLGLLGPLAILLALY
CD3ζ Transmembrane domain
SEQ ID NO: 36
DLCYLLDGILFIYGVILTALFLRVK
CD3ε Transmembrane domain
SEQ ID NO: 37
DVMSVATIVIVDICITGGLLLLVYYWSKN
CD3γ Transmembrane domain
SEQ ID NO: 38
DGFLFAEIVSIFVLAVGVYFIAGQD
CD3δ Transmembrane domain
SEQ ID NO: 39
DGIIVTDVIATLLLALGVFCFAGHET
TCR-α Transmembrane domain
SEQ ID NO: 40
DVIGFRILLLKVAGFNLLMTLRLW
CD32 Transmembrane domain
SEQ ID NO: 41
DIIVAVVIATAVAAIVAAVVALIYCRK
CD64 Transmembrane domain
SEQ ID NO: 42
DVLFYLAVGIMFLVNTVLWVTIRKE
VEFGR2 Transmembrane domain
SEQ ID NO: 43
DIIILVGTAVIAMFFWLLLVIILRT
FAS Transmembrane domain
SEQ ID NO: 44
DLGWLCLLLLPIPLIVWVKRK
FGFR2B Transmembrane domain
SEQ ID NO: 45
DIAIYCIGVFLIACMVVTVILCRMK

### REFERENCES

Abaricia, S., & Hirbe, A. C. (2018). Diagnosis and Treatment of Myxoid Liposarcomas: Histology Matters. Curr Treat Options Oncol, 19(12), 64.
Aguiar, S., Dias, J., Manuel, A. M., Russo, R., Gois, P. M. P., da Silva, F. A., & Goncalves, J. (2018). Chimeric Small Antibody Fragments as Strategy to Deliver Therapeutic Payloads. Adv Protein Chem Struct Biol, 112, 143-182.
Amer, K. M., Congiusta, D. V., Thomson, J. E., Elsamna, S., Chaudhry, I., Bozzo, A., Amer, R., Siracuse, B., Ghert, M., & Beebe, K. S. (2020). Epidemiology and survival of liposarcoma and its subtypes: A dual database analysis. J Clin Orthop Trauma, 11(Suppl 4), 5479-s484.
Arriga, R., Caratelli, S., Lanzilli, G., Ottaviani, A., Cenciarelli, C., Sconocchia, T., Spagnoli, G. C., Iezzi, G., Roselli, M., Lauro, D., Coppola, A., Dotti, G., Ferrone, S., & Sconocchia, G. (2020). CD16-158-valine chimeric receptor T cells overcome the resistance of KRAS-mutated colorectal carcinoma cells to cetuximab. Int J Cancer, 146(9), 2531-2538.
Asano, N., Susa, M., Hosaka, S., Nakayama, R., Kobayashi, E., Takeuchi, K., Horiuchi, K., Suzuki, Y., Anazawa, U., Mukai, M., Toyama, Y., Yabe, H., & Morioka, H. (2012). Metastatic patterns of myxoid/round cell liposarcoma: a review of a 25-year experience. Sarcoma, 2012, 345161-345161.
Carmeliet, P., & Jain, R. K. (2000). Angiogenesis in cancer and other diseases. Nature, 407(6801), 249-257.
Chen, X., Higgins, J., Cheung, S. T., Li, R., Mason, V., Montgomery, K., Fan, S. T., van de Rijn, M., & So, S. (2004). Novel endothelial cell markers in hepatocellular carcinoma. Mod Pathol, 17(10), 1198-1210.
Cree, I. A., Uttley, L., Buckley Woods, H., Kikuchi, H., Reiman, A., Harnan, S., Whiteman, B. L., Philips, S. T., Messenger, M., Cox, A., Teare, D., Sheils, O., & Shaw, J. (2017). The evidence base for circulating tumour DNA blood-based biomarkers for the early detection of cancer: a systematic mapping review. BMC Cancer, 17(1), 697.
David, G. S., & Reisfeld, R. A. (1974). Protein iodination with solid state lactoperoxidase. Biochemistry, 13(5), 1014-1021.
Dodd, L. G. (2012). Update on liposarcoma: a review for cytopathologists. Diagn Cytopathol, 40(12), 1122-1131.
Fritchie, K. J., Goldblum, J. R., Tubbs, R. R., Sun, Y., Carver, P., Billings, S. D., & Rubin, B. P. (2012). The Expanded Histologic Spectrum of Myxoid Liposarcoma With an Emphasis on Newly Described Patterns: Implications for Diagnosis on Small Biopsy Specimens. American Journal of Clinical Pathology, 137(2), 229-239.
Ge, Z., Helmijr, J. C. A., Jansen, M. P. H. M., Boor, P. P. C., Noordam, L., Peppelenbosch, M., Kwekkeboom, J., Kraan, J., & Sprengers, D. (2021). Detection of oncogenic mutations in paired circulating tumor DNA and circulating tumor cells in patients with hepatocellular carcinoma. Translational Oncology, 14(7), 101073.
Giepmans, B. N., Adams, S. R., Ellisman, M. H., & Tsien, R. Y. (2006). The fluorescent toolbox for assessing protein location and function. Science, 312(5771), 217-224.
Haniball, J., Sumathi, V. P., Kindblom, L. G., Abudu, A., Carter, S. R., Tillman, R. M., Jeys, L., Spooner, D., Peake, D., & Grimer, R. J. (2011). Prognostic Factors and Metastatic Patterns in Primary Myxoid/Round-Cell Liposarcoma. Sarcoma, 2011, 538085.
Hemminger, J. A., & Iwenofu, O. H. (2013). NY-ESO-1 is a sensitive and specific immunohistochemical marker for myxoid and round cell liposarcomas among related mesenchymal myxoid neoplasms. Mod Pathol, 26(9), 1204-1210.
Henze, J., & Bauer, S. (2013). Liposarcomas. Hematol Oncol Clin North Am, 27(5), 939-955.
Hippo, Y., Watanabe, K., Watanabe, A., Midorikawa, Y., Yamamoto, S., Ihara, S., Tokita, S., Iwanari, H., Ito, Y., Nakano, K., Nezu, J., Tsunoda, H., Yoshino, T., Ohizumi, I., Tsuchiya, M., Ohnishi, S., Makuuchi, M., Hamakubo, T., Kodama, T., & Aburatani, H. (2004). Identification of soluble NH2-terminal fragment of glypican-3 as a serological marker for early-stage hepatocellular carcinoma. Cancer Res, 64(7), 2418-2423.
Ho, M., & Kim, H. (2011). Glypican-3: a new target for cancer immunotherapy. Eur J Cancer, 47(3), 333-338.
Hui, J. Y. (2016). Epidemiology and Etiology of Sarcomas. Surg Clin North Am, 96(5), 901-914.
Jena, B., Dotti, G., & Cooper, L. J. (2010). Redirecting T-cell specificity by introducing a tumor-specific chimeric antigen receptor. Blood, 116(7), 1035-1044.
Jo, V. Y., & Fletcher, C. D. M. (2014). WHO classification of soft tissue tumours: an update based on the 2013 (4th) edition. Pathology, 46(2), 95-104.
Kim, J. H., Lee, S. R., Li, L. H., Park, H. J., Park, J. H., Lee, K. Y., Kim, M. K., Shin, B. A., & Choi, S. Y. (2011). High cleavage efficiency of a 2A peptide derived from porcine teschovirus-1 in human cell lines, zebrafish and mice. PLoS One, 6(4), e18556.
Kuo, M. L., Sutkowski, N., Ron, Y., & Dougherty, J. P. (1993). Efficient gene transfer into primary murine lymphocytes obviating the need for drug selection. Blood, 82(3), 845-852.
Lee, A. T. J., Thway, K., Huang, P. H., & Jones, R. L. (2018). Clinical and Molecular Spectrum of Liposarcoma. J Clin Oncol, 36(2), 151-159.
Leong, A. S. (1996). Microwaves in diagnostic immunohistochemistry. Eur J Morphol, 34(5), 381-383.
Low, W. S., & Wan Abas, W. A. (2015). Benchtop technologies for circulating tumor cells separation based on biophysical properties. Biomed Res Int, 2015, 239362.
Lu, S. X., Huang, Y. H., Liu, L. L., Zhang, C. Z., Yang, X., Yang, Y. Z., Shao, C. K., Li, J. M., Xie, D., Zhang, X., Jain, D., & Yun, J. P. (2021). α-Fetoprotein mRNA in situ hybridisation is a highly specific marker of hepatocellular carcinoma: a multi-centre study. Br J Cancer, 124(12), 1988-1996.
Mann, R., Mulligan, R. C., & Baltimore, D. (1983). Construction of a retrovirus packaging mutant and its use to produce helper-free defective retrovirus. Cell, 33(1), 153-159.
Maravelia, P., Silva, D. N., Rovesti, G., Chrobok, M., Stål, P., Lu, Y. C., & Pasetto, A. (2021). Liquid Biopsy in Hepatocellular Carcinoma: Opportunities and Challenges for Immunotherapy. Cancers (Basel), 13(17).
Markowitz, D., Goff, S., & Bank, A. (1988). A safe packaging line for gene transfer: separating viral genes on two different plasmids. J Virol, 62(4), 1120-1124.
Nygren, H. (1982). Conjugation of horseradish peroxidase to Fab fragments with different homobifunctional and heterobifunctional cross-linking reagents. A comparative study. J Histochem Cytochem, 30(5), 407-412.
O'Sullivan, M. J., & Marks, V. (1981). Methods for the preparation of enzyme-antibody conjugates for use in enzyme immunoassay. Methods Enzymol, 73(Pt B), 147-166.
Ono, A., Fujimoto, A., Yamamoto, Y., Akamatsu, S., Hiraga, N., Imamura, M., Kawaoka, T., Tsuge, M., Abe, H., Hayes, C. N., Miki, D., Furuta, M., Tsunoda, T., Miyano, S., Kubo, M., Aikata, H., Ochi, H., Kawakami, Y. I., Arihiro, K., Ohdan, H., Nakagawa, H., & Chayama, K. (2015). Circulating Tumor DNA Analysis for Liver Cancers and Its Usefulness as a Liquid Biopsy. Cell Mol Gastroenterol Hepatol, 1(5), 516-534.
Pain, D., & Surolia, A. (1981). Preparation of protein A-peroxidase monoconjugate using a heterobifunctional reagent, and its use in enzyme immunoassays. J Immunol Methods, 40(2), 219-230.
Patil, M. A., Chua, M. S., Pan, K. H., Lin, R., Lih, C. J., Cheung, S. T., Ho, C., Li, R., Fan, S. T., Cohen, S. N., Chen, X., & So, S. (2005). An integrated data analysis approach to characterize genes highly expressed in hepatocellular carcinoma. Oncogene, 24(23), 3737-3747.
Ponziani, S., Di Vittorio, G., Pitari, G., Cimini, A. M., Ardini, M., Gentile, R., Iacobelli, S., Sala, G., Capone, E., Flavell, D. J., Ippoliti, R., & Giansanti, F. (2020). Antibody-Drug Conjugates: The New Frontier of Chemotherapy. Int J Mol Sci, 21(15).
Regina, C., & Hettmer, S. (2019). Myxoid liposarcoma: it's a hippo's world. EMBO Mol Med, 11(5).
Sanfilippo, R., Dei Tos, A. P., & Casali, P. G. (2013). Myxoid liposarcoma and the mammalian target of rapamycin pathway. Current Opinion in Oncology, 25(4).
Shank, B. R., Do, B., Sevin, A., Chen, S. E., Neelapu, S. S., & Horowitz, S. B. (2017). Chimeric Antigen Receptor T Cells in Hematologic Malignancies. Pharmacotherapy, 37(3), 334-345.
Singhi, A. D., & Montgomery, E. A. (2011). Liposarcomas. Surg Pathol Clin, 4(3), 963-994.
Suarez-Kelly, L. P., Baldi, G. G., & Gronchi, A. (2019). Pharmacotherapy for liposarcoma: current state of the art and emerging systemic treatments. Expert Opinion on Pharmacotherapy, 20(12), 1503-1515.
Wang, F., Flanagan, J., Su, N., Wang, L. C., Bui, S., Nielson, A., Wu, X., Vo, H. T., Ma, X. J., & Luo, Y. (2012). RNAscope: a novel in situ RNA analysis platform for formalin-fixed, paraffin-embedded tissues. J Mol Diagn, 14(1), 22-29.
Wang, S., Chen, N., Chen, Y., Sun, L., Li, L., & Liu, H. (2018). Elevated GPC3 level promotes cell proliferation in liver cancer. Oncol Lett, 16(1), 970-976.
Wang, Y., Shen, Z., Zhu, Z., Han, R., & Huai, M. (2011). Clinical values of AFP, GPC3 mRNA in peripheral blood for prediction of hepatocellular carcinoma recurrence following OLT: AFP, GPC3 mRNA for prediction of HCC. Hepat Mon, 11(3), 195-199.
Weber, W. A. (2009). Assessing tumor response to therapy. J Nucl Med, 50 Suppl 1, is-10s.
Yi, B., Wu, T., Zhu, N., Huang, Y., Yang, X., Yuan, L., Wu, Y., Liang, X., & Jiang, X. (2021). The clinical significance of CTC enrichment by GPC3-IML and its genetic analysis in hepatocellular carcinoma. J Nanobiotechnology, 19(1), 74.
Zhang, J., Campbell, R. E., Ting, A. Y., & Tsien, R. Y. (2002). Creating new fluorescent probes for cell biology. Nature Reviews Molecular Cell Biology, 3(12), 906-918.
Zhang, J., & Wang, L. (2019). The Emerging World of TCR-T Cell Trials Against Cancer: A Systematic Review. Technol Cancer Res Treat, 18, 1533033819831068.
Zhou, F., Shang, W., Yu, X., & Tian, J. (2018). Glypican-3: A promising biomarker for hepatocellular carcinoma diagnosis and treatment. Med Res Rev, 38(2), 741-767.

### OTHER EMBODIMENTS

All the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method of treating a patient diagnosed with myxoid/round cell liposarcoma, the method comprising administering an anti-Glypican-3 (GPC3) therapeutic agent to the patient.

2. The method of claim 1, wherein the patient is selected for treatment by diagnosing myxoid/round cell liposarcoma.

3. The method of claim 1, wherein the myxoid/round cell liposarcoma expresses GPC3.

4. The method of claim 1, wherein the patient diagnosed with the myxoid/round cell liposarcoma is selected by immunostaining, optionally by immunohistochemistry (IHC) staining, preferably with a cytoplasmic/membranous H-score of greater than 30.

5. The method of claim 1, wherein the patient is diagnosed by a process comprising:
(a) obtaining a tissue section from a tumor biopsy sample, the section having a thickness between 3 μm and 15 μm,
(b) immunostaining preferably by IHC with an antibody that specifically binds to GPC3, more specifically using the antibody GC33,
(c) determining the cytoplasmic/membranous H-score, and
(d) selecting patients with an H-score of greater than 30 for the treatment.

6. The method of claim 1, wherein the selection comprises immunostaining, preferably immunohistochemical staining of GPC3 in a tumor sample from the patient, wherein the GPC3 expression level is determined and compared to a predetermined threshold level of GPC3 expression, and wherein the patient is selected for treatment in case the patient has a GPC3 expression level equal or higher to the predetermined threshold level.

7. The method of claim 1, wherein the therapeutic agent comprises an anti-GPC3 binding domain, preferably an anti-GPC3 antibody or functional fragment thereof retaining binding to GPC3, preferably wherein the therapeutic agent comprises the administration of an anti-GPC3 antibody, an anti-GPC3 antibody-drug conjugate, an anti-GPC3 antibody-radionuclide conjugate, or a fusion protein of an anti-GPC3 antibody or antibody derivative binding to GPC3 with an anti-CD3 binding domain or an immunostimulatory polypeptide.

8. The method of claim 1, wherein the therapeutic agent comprises the administration of genetically engineered hematopoietic cells expressing an anti-GPC3 chimeric receptor polypeptide (CAR), wherein the CAR polypeptide comprises:
(a) an extracellular binding domain binding to GPC3;
(b) a transmembrane domain; and
(c) a cytoplasmic signaling domain,
and optionally wherein the hematopoietic cell exogenously expresses a gene that improves viability and/or functionality of the hematopoietic cell in the solid tumor microenvironment.

9. The method of claim 8, wherein the hematopoietic cells have
i. an improved glucose uptake activity as relative to a wild-type hematopoietic cell of the same type, whereas the hematopoietic cell exogenously expresses a glucose importation polypeptide, preferably wherein the glucose importation polypeptide is a glucose transporter (GLUT) or a sodium-glucose cotransporter (SGLT), preferably wherein the glucose importation polypeptide is selected from the group consisting of: GLUT1, GLUT3, GLUT1 S226D, SGLT1, SGLT2, GLUT8, GLUT8 L12A L13A, GLUT11, GLUT7, and GLUT4;
ii. a modulated Krebs cycle as relative to a wild-type hematopoietic cell of the same type, whereas the hematopoietic cell exogenously expresses a Krebs cycle modulating polypeptide,
preferably wherein the Krebs cycle modulating factor is
a. an enzyme that catalyzes a reaction in the Krebs cycle, preferably isocitrate dehydrogenase (IDH), malate dehydrogenase (MDH), or phosphoglycerate dehydrogenase (PHGDH),
b. an enzyme that uses a Krebs cycle metabolite as a substrate, preferably glutamic-oxaloacetic transaminase (GOT) or phosphoenolpyruvate carboxykinase 1 (PCK1), or
c. an enzyme that converts a precursor to a Krebs cycle metabolite, preferably phosphoserine aminotransferase (PSAT1), glutamate dehydrogenase (GDH1), glutamate-pyruvate transaminase 1 (GPT1), or glutaminase (GLS); and/or
iii. enhanced intracellular lactate concentrations relative to a wild-type hematopoietic cell of the same type, whereas the hematopoietic cell exogenously expresses a lactate-modulating polypeptide,
preferably wherein the lactate modulation polypeptide is
a. monocarboxylate transporter (MCT), preferably MCT1, MCT2, or MCT4,
b. an enzyme involved in lactate synthesis, preferably lactate dehydrogenase A (LDHA), or
c. a polypeptide that inhibits a pathway that competes for lactate-synthesis substrates, preferably pyruvate dehydrogenase kinase 1 (PDK1).

10. The method of claim 8, wherein the extracellular antigen binding domain is a single chain antibody fragment (scFv) that binds to a GPC3, preferably wherein the scFv is derived from the GC33 antibody, optionally wherein the scFv has the sequence of SEQ ID NO: 2.

11. The method of claim 8, wherein the CAR polypeptide comprises:
(i) a CD28 co-stimulatory domain, in combination with a CD28 transmembrane domain, preferably SEQ ID NO: 6, a CD28 hinge domain, or a combination thereof, preferably SEQ ID NO: 4 or
(ii) a 4-1BB co-stimulatory domain, preferably SEQ ID NO: 5, in combination with a CD8 transmembrane domain, a CD8 hinge domain, or a combination thereof (SEQ ID NO: 3);
more preferably wherein the CAR polypeptide comprises the amino acid sequence of SEQ ID NO: 8 or SEQ ID NO: 9.

12. The method of claim 8, wherein the cytoplasmic signaling domain of (c) is a cytoplasmic domain of CD3ζ, preferably SEQ ID NO: 7, or FcεR1γ.

13. The method of claim 8, wherein the hematopoietic cells are natural killer (NK) cells, macrophages, neutrophils, eosinophils, or T cells, preferably wherein the hematopoietic cells are T cells, in which the expression of an endogenous T cell receptor, an endogenous major histocompatibility complex, an endogenous beta-2-microglobulin, or a combination thereof has been inhibited or eliminated; and/or wherein the hematopoietic cells are derived from peripheral blood mononuclear cells (PBMC), hematopoietic stem cells (HSCs), or inducible pluripotent stem cells (iPSCs), preferably wherein the hematopoietic cells are autologous to the patient.

14. The method of claim 8, wherein the hematopoietic cells comprise a nucleic acid or a set of nucleic acids, preferably a DNA molecule or a set of DNA molecules, which collectively comprises:
(a) a first nucleotide sequence encoding the glucose importation polypeptide, the Krebs cycle modulating polypeptide and/or the lactate-modulating polypeptide; and
(b) a second nucleotide sequence encoding the chimeric antigen receptor polypeptide; preferably wherein the hematopoietic cells comprise the nucleic acid, which comprises both the first nucleotide sequence and the second nucleotide sequence; and
(c) optionally a third nucleotide sequence located between the first nucleotide sequence and the second nucleotide sequence, wherein the third nucleotide sequence encodes a ribosomal skipping site, an internal ribosome entry site (IRES), or a second promoter, preferably wherein the third nucleotide sequence encodes a ribosomal skipping site, which is a P2A peptide;
preferably wherein:
(i) the nucleic acid or the nucleic acid set is comprised within a vector or a set of vectors, which preferably is an expression vector or a set of expression vectors; and/or
(ii) the vector or set of vectors comprises one or more viral vectors, which more preferably is a lentiviral vector or retroviral vector.

15. The method of claim 1, wherein at least about 5×10⁴ anti-GPC3−CAR T cells per kg are administered to the patient, preferably from about 5×10⁴ to about 1×10¹² anti-GPC3-CAR T cells/kg are administered to the patient.

16. The method of claim 1, wherein the therapeutic agent comprises an anti-GPC3 targeted polypeptide or polypeptide fusion, preferably an anti-GPC3 antibody, an anti-GPC3 bi- or multiple specific protein or an anti-GPC3 antibody-drug-conjugate.

17. The method of claim 1, wherein the administration of the anti-GPC3 therapeutic agent is effective in achieving stable disease according to RECIST as measured by computerized tomography (CT) scan.

18. The method of claim 1, wherein the administration of the anti-GPC3 therapeutic agent is achieving an objective response according to RECIST as measured by computerized tomography (CT) scan.

19. The method of claim 1, wherein the method further comprises administering at least one immunomodulatory agent to the patient in parallel or sequential to the therapeutic agent, preferably where in the immunomodulatory agent is an immune checkpoint inhibitor or an immunostimulatory cytokine.

20. The method of claim 1, wherein the method further comprises administering to the subject a lymphocyte reduction treatment, preferably selected from cyclophosphamide and fludarabine.


SEQUENCE LISTING (45 sequences)

SEQ ID NO: 1 - protein, 21 aa, synthetic construct
MALPVTALLLPLALLLHAARP

SEQ ID NO: 2 - protein, 243 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSS

SEQ ID NO: 3 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 4 - protein, 107 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 5 - protein, 42 aa, synthetic construct
KRGRKKLLYIFKQPFMRPVQTTQEEDGCSCRFPEEEEGGCEL

SEQ ID NO: 6 - protein, 41 aa, synthetic construct
RSKRSRLLHSDYMNMTPRRPGPTRKHYQPYAPPRDFAAYRS

SEQ ID NO: 7 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 487 aa, synthetic construct
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 466 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 483 aa, synthetic construct
MALPVTALLLPLALLLHAARPDVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 462 aa, synthetic construct
DVVMTQSPLSLPVTPGEPASISCRSSQSLVHSNRNTYLHWYLQKPGQSPQLLIYKVSNRFSGVPDRFSGSGSGTDFTLKISRVEAEDVGVYYCSQNTHVPPTFGQGTKLEIKRGGGGSGGGGSGGGGSQVQLVQSGAEVKKPGASVKVSCKASGYTFTDYEMHWVRQAPGQGLEWMGALDPKTGDTAYSQKFKGRVTLTADKSTSTAYMELSSLTSEDTAVYYCTRFYSYTYWGQGTLVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 430 aa, synthetic construct
MALLHSGRVLPGIAAAFHPGLAAAASARASSWWTHVEMGPPDPILGVTEAFKRDTNSKKMNLGVGAYRDDNGKPYVLPSVRKAEAQIAAKNLDKEYLPIGGLAEFCKASAELALGENSEVLKSGRFVTVQTISGTGALRIGASFLQRFFKFSRDVFLPKPTWGNHTPIFRDAGMQLQGYRYYDPKTCGFDFTGAVEDISKIPEQSVLLLHACAHNPTGVDPRPEQWKEIATVVKKRNLFAFFDMAYQGFASGDGDKDAWAVRHFIEQGINVCLCQSYAKNMGLYGERVGAFTMVCKDADEAKRVESQLKILIRPMYSNPPLNGARIAAAILNTPDLRKQWLQEVKVMADRIIGMRTQLVSNLKKEGSTHNWQHITDQIGMFCFTGLKPEQVERLIKEFSIYMTKDGRISVAGVTSSNVGYLAHAIHQVTK

SEQ ID NO: 13 - protein, 413 aa, synthetic construct
MAPPSVFAEVPQAQPVLVFKLTADFREDPDPRKVNLGVGAYRTDDCHPWVLPVVKKVEQKIANDNSLNHEYLPILGLAEFRSCASRLALGDDSPALKEKRVGGVQSLGGTGALRIGADFLARWYNGTNNKNTPVYVSSPTWENHNAVFSAAGFKDIRSYRYWDAEKRGLDLQGFLNDLENAPEFSIVVLHACAHNPTGIDPTPEQWKQIASVMKHRFLFPFFDSAYQGFASGNLERDAWAIRYFVSEGFEFFCAQSFSKNFGLYNERVGNLTVVGKEPESILQVLSQMEKIVRITWSNPPAQGARIVASTLSNPELFEEWTGNVKTMADRILTMRSELRARLEALKTPGTWNHITDQIGMFSFTGLNPKQVEYLVNEKHIYLLPSGRINVSGLTTKNLDYVATSIHEAVTKIQ

SEQ ID NO: 14 - protein, 19 aa, synthetic construct
ATNFSLLKQAGDVEENPGP

SEQ ID NO: 15 - protein, 488 aa, synthetic construct
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIWGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAWLQLSQQLSGINAVFYYSTSIFEKAGVQQPVYATIGSGIVNTAFTWSLFWERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 16 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 17 - protein, 238 aa, synthetic construct
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 18 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 19 - protein, 238 aa, synthetic construct
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 20 - protein, 42 aa, synthetic construct
ALYLLRRDQRLPPDAHKPPGGGSFRTPIQEEQADAHSTLAKI

SEQ ID NO: 21 - protein, 48 aa, synthetic construct
QRRKYRSNKGESPVEPAEPCHYSCPREEEGSTIPIQEDYRKPEPACSP

SEQ ID NO: 22 - protein, 34 aa, synthetic construct
KKKYSSSVHDPNGEYMFMRAVNTAKKSRLTDVTL

SEQ ID NO: 23 - protein, 58 aa, synthetic construct
QLGLHIWQLRSQCMWPRETQLLLEVPPSTEDARSCQFPEEERGERSAEEKGRLGDLWV

SEQ ID NO: 24 - protein, 60 aa, synthetic construct
CVKRRKPRGDVVKVIVSVQRKRQEAEGEATVIEALQAPPDVTTVAVEETIPSFTGRSPNH

SEQ ID NO: 25 - protein, 48 aa, synthetic construct
KKYFFKKEVQQLSVSFSSLQIKALQNAVEKEVQAEDNIYIENSLYATD

SEQ ID NO: 26 - protein, 84 aa, synthetic construct
DIYIWAPLAGTCGVLLLSLVITLYCYKVGFFKRNLKEKMEAGRGVPNGIPAEDSEQLASGQEAGDPGCLKPLHEKDSESGGGKD

SEQ ID NO: 27 - protein, 141 aa, synthetic construct
DIYIWAPLAGTCGVLLLSLVITLYCKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 28 - protein, 25 aa, synthetic construct
DIYIWAPLAGTCGVLLLSLVITLYC

SEQ ID NO: 29 - protein, 25 aa, synthetic construct
DITLGLLVAGVLVLLVSLGVAIHLC

SEQ ID NO: 30 - protein, 28 aa, synthetic construct
DIISFFLALTSTALLFLLFFLTLRFSVV

SEQ ID NO: 31 - protein, 30 aa, synthetic construct
DFWVLVVVGGVLACYSLLVTVAFIIFWVRS

SEQ ID NO: 32 - protein, 25 aa, synthetic construct
DLIALVTSGALLAVLGITGYFLMNR

SEQ ID NO: 33 - protein, 26 aa, synthetic construct
DMALIVLGGVAGLLLFIGLGIFFCVR

SEQ ID NO: 34 - protein, 25 aa, synthetic construct
DLCYILDAILFLYGIVLTLLYCRLK

SEQ ID NO: 35 - protein, 25 aa, synthetic construct
DVAAILGLGLVLGLLGPLAILLALY

SEQ ID NO: 36 - protein, 25 aa, synthetic construct
DLCYLLDGILFIYGVILTALFLRVK

SEQ ID NO: 37 - protein, 29 aa, synthetic construct
DVMSVATIVIVDICITGGLLLLVYYWSKN

SEQ ID NO: 38 - protein, 25 aa, synthetic construct
DGFLFAEIVSIFVLAVGVYFIAGQD

SEQ ID NO: 39 - protein, 26 aa, synthetic construct
DGIIVTDVIATLLLALGVFCFAGHET

SEQ ID NO: 40 - protein, 24 aa, synthetic construct
DVIGFRILLLKVAGFNLLMTLRLW

SEQ ID NO: 41 - protein, 27 aa, synthetic construct
DIIVAVVIATAVAAIVAAVVALIYCRK

SEQ ID NO: 42 - protein, 25 aa, synthetic construct
DVLFYLAVGIMFLVNTVLWVTIRKE

SEQ ID NO: 43 - protein, 25 aa, synthetic construct
DIIILVGTAVIAMFFWLLLVIILRT

SEQ ID NO: 44 - protein, 21 aa, synthetic construct
DLGWLCLLLLPIPLIVWVKRK

SEQ ID NO: 45 - protein, 25 aa, synthetic construct
DIAIYCIGVFLIACMVVTVILCRMK